(19) United States
(12) Patent Application Publication
(10) Pub. No.: US 2026/0309723 A1
(43) Pub. Date: Oct. 8, 2026

(54) EARLY DETECTION AND MONITORING OF NEURODEGENERATIVE DISEASES USING A MULTIDISEASE DIAGNOSTIC PLATFORM

(71) Applicant: Durin Life Sciences, Inc., Stratford, NJ (US)

(72) Inventors: Cassandra DeMarshall, Voorhees, NJ (US); Jeffrey Viviano, Voorhees, NJ (US)

(73) Assignee: Durin Life Sciences, Inc., Stratford, NJ (US)

(21) Appl. No.: 18/998,436

(22) PCT Filed: Jul. 27, 2023
(86) PCT No.: PCT/US23/71129

§ 371 Date: Jan. 24, 2025

Related U.S. Application Data
(60) Provisional application No. 63/369,561, filed on Jul. 27, 2022.

(51) Int. Cl. G01N 33/68; G01N 33/564; G01N 33/58; G16B 40/20; G16H 50/20; G16H 70/60

(52) U.S. Cl. CPC G01N 33/6896; G01N 33/564; G01N 33/582; G16B 40/20; G16H 50/20; G16H 70/60; G01N 2800/2821; G01N 2800/2835

(57) ABSTRACT

Methods, systems and kits useful for the detection and diagnosis of neurodegenerative diseases including Alzheimer's Disease (AD)- and early-stage Parkinson's Disease-related pathology, and methods of preparing labeled immunocomplexes useful for detecting AD- and PD-related pathology are provided.

### CROSS-REFERENCE TO RELATED APPLICATIONS

The present application claims priority under 35 U.S.C. § 119(e) to the U.S. Provisional Patent Application No. 63/369,561, filed Jul. 27, 2022. The entire disclosure of the application noted above is incorporated herein by reference.

### FIELD OF THE INVENTION

The invention relates generally to the fields of medicine, neurology and molecular biology. In particular, the invention relates to methods, systems and kits for early detection (diagnosis) and monitoring of neurodegenerative diseases and disease pathology, and methods of preparing labeled immunocomplexes useful for detecting neurodegenerative diseases and disease pathology.

### REFERENCE TO A SEQUENCE LISTING

This application incorporates by reference the Sequence Listing submitted in Computer Readable Form as file SeqList_100561-00033, created on Jul. 24, 2023 and containing 14,687 bytes.

### BACKGROUND

Neurodegenerative diseases are disorders of the central nervous system that affect millions of people worldwide. Characterized by loss of neuronal function, ultimately resulting in cellular death, neurodegenerative diseases are hallmarked by progressive and incurable symptoms including motor impairment and dementia. Because the prevalence and incidence of these diseases increases with advancing age, Alzheimer's (AD) and Parkinson's diseases (PD) are the most common neurodegenerative diseases, but they also include amyotrophic lateral sclerosis, multiple sclerosis, multiple system atrophy, and Huntington's disease, among others. Although neurodegenerative diseases pose a significant medical and public health burden, the majority of these diseases lack simple and accurate tests for the diagnosis of individuals in the earliest stages of the diseases.

Alzheimer's Disease (AD) is a devastating, neurodegenerative disease affecting roughly 6 million people in the US. AD pathology is now known to begin a decade or more before emergence of its hallmark symptoms, rendering early diagnosis a challenge. This implies that, by the time telltale symptoms emerge and can be used to diagnose AD using neuropsychological assessments and initiate treatments, a considerable amount of brain devastation may already have occurred, making it difficult to slow, stop or potentially reverse the disease. Traditional methods to diagnose AD most often involve a clinical judgement made by weighing data derived from some combination of patient history, a wide variety of simple and more extensive neuropsychological screeners and tests, diagnostic imaging, and cerebrospinal fluid (CSF) analysis of various biomarkers, such as Aβ42 and Aβ40, total Tau, and various forms of phosphorylated Tau (pTau). While some of these methods are currently the “gold standard” for AD diagnosis, they are expensive, invasive, require highly skilled personnel to perform and evaluate these tests, and are largely inaccessible to most people throughout the world.

Parkinson's disease (PD) is a chronic and progressive motor system disorder inflicting profound social and economic costs worldwide. It is the second most common neurodegenerative disorder after AD, affecting more than 1% of 55-year-old individuals and more than 3% of those over the age of 75. The primary symptoms of PD include tremor, rigidity, bradykinesia, and postural instability. The cardinal pathological feature of PD is the loss of dopaminergic neurons in the substantia nigra, a brain region involved in coordination and control of muscle activity. Although PD manifests primarily as a motor disability, recent studies reveal many pre-motor symptoms that suggest an onset of PD pathology years before characteristic symptoms appear. By the time a diagnosis is made, at least one-third of substantia nigra neurons and striatal dopaminergic fibers are already lost. Despite years of research, there is no one test or technique that can provide a conclusive primary diagnosis of PD. Current diagnostic methods are based on medical history evaluation and a combination of physical and neurological assessments. Standard practices for these assessments, such as the Unified Parkinson's Disease Rating Scale (UPDRS), have aided tremendously in clinical staging of the disease, but fail to detect PD before the onset of initial motor symptoms. Additional techniques, such as CT, MRI, and PET neuroimaging, may be used to rule out other neurological disorders, but rarely do they detect any abnormality that can be directly related to the onset of PD. There are also no laboratory tests utilizing blood, cerebrospinal fluid, or urine samples that have proven to be effective in primary diagnosis or confirmation of PD.

Although potential diagnostic tests are under development, no FDA-approved blood or laboratory tests for AD or PD exist that provide a diagnosis during pre-symptomatic and MCI stages of AD, and early-stages of PD, or assist in monitoring subsequent progression of these diseases. Pre-symptomatic and prodromal AD, as well as early-stage PD, have been particularly difficult to diagnose using current methods. Thus, the development of accurate, noninvasive, blood-based diagnostic tests for early AD and PD detection and monitoring for use in primary care or other frontline settings is essential to implement early treatment. In addition, such advancements would enhance the tracking of AD and PD neuropathological and cognitive progression, facilitate earlier participation in clinical trials, and inform interventions to combat these highly prevalent diseases.

### SUMMARY OF THE INVENTION

Described herein are methods, systems, kits and platforms for detection (diagnosis) and monitoring of neurodegenerative diseases such as AD and PD, including detection in early stages of the diseases. In one embodiment, the present disclosure provides a method for detecting AD-related pathology in a subject, the method including: (a) obtaining an immunoglobulin-containing biological sample from the subject; (b) providing a protein target set including protein targets MGC31944 or MGC31936, HSH2D, GCDH, CCL19, LGALS1 (Galectin-1), DNAJC8, ICAM-4, and anti-human anti-IgG kappa light chain antibody, and/or epitope binding fragments thereof, wherein the protein targets and/or epitope binding fragments thereof are attached to a substrate, (c) contacting the biological sample with the protein target set under conditions under which each of MGC31944 or MGC31936, HSH2D, GCDH, CCL19, LGALS1 (Galectin-1), DNAJC8, and ICAM-4, or epitope binding fragments thereof, forms an immunocomplex with a corresponding AD-related autoantibody biomarker if present in the biological sample, and the anti-human anti-IgG kappa light chain antibody forms immunocomplexes with all IgG antibodies having a kappa light chain in the biological sample; (d) adding a detectable label to the immunocomplexes such that the immunocomplexes are labeled; (e) measuring the levels of the labeled immunocomplexes to generate levels of AD-related autoantibody biomarkers and a total IgG antibody level in the biological sample; (f) obtaining at least one covariate data set generated from the subject; and (g) determining an AD score for the subject by inputting the levels of the subject's AD-related autoantibody biomarkers, total IgG antibody level, and the covariate data into a classification algorithm configured to (1) determine therefrom an AD score, and (2) output the AD score. In embodiments, the method can also include one or more of: analyzing at least one covariate of the subject to generate covariate data, receiving the AD score and detecting the presence of AD-related pathology in a subject with early stage AD if the subject's AD score is equal to or greater than a predetermined threshold score at or above which there is a likelihood that a subject has ongoing AD-related pathology. In these methods the protein target set is used in the detection and quantification of the levels of AD-related autoantibodies and titers of IgG in the biological sample.

Also described herein is a method for detecting the presence of PD-related pathology in a subject with early-stage PD, the method including: (a) obtaining an immunoglobulin-containing biological sample from the subject; (b) providing a protein target set including protein targets MARK1, PUSL1, IL20, CCL19, and anti-human anti-IgG kappa light chain antibody, and/or epitope binding fragments thereof, wherein the protein targets and/or epitope binding fragments thereof are attached to a substrate, (c) contacting the biological sample with the protein target set under conditions under which each of MARK1, PUSL1, IL20, CCL19, or epitope binding fragments thereof, forms an immunocomplex with a corresponding PD-related autoantibody biomarker if present in the biological sample, and the anti-human anti-IgG kappa light chain antibody forms immunocomplexes with all IgG antibodies having a kappa light chain in the biological sample; (d) adding a detectable label to the immunocomplexes such that the immunocomplexes are labeled; (e) measuring the levels of the labeled immunocomplexes to generate levels of PD-related autoantibody biomarkers and a total IgG antibody level in the biological sample; (f) obtaining at least one covariate data set generated from the subject; and (g) determining a PD score for the subject by inputting the levels of the subject's PD autoantibody biomarker, total IgG antibody level, and the covariate data into a classification algorithm configured to (1) determine therefrom an PD score, and (2) output the PD score. In embodiments, the method can also include one or more of: analyzing at least one covariate of the subject to generate covariate data, receiving the PD score, and detecting that the subject has early-stage PD-related pathology if the subject's PD score is equal to or greater than a predetermined threshold score at or above which there is a likelihood that a subject has ongoing PD-related pathology. In these methods the protein target set is used in the detection and quantification of the levels of PD-related autoantibodies in the biological sample.

In embodiments of the methods, the biological sample is blood or serum. The substrate can include a plurality of beads, each bead attached to only one type of protein target, and/or epitope binding fragment thereof, of the protein targets. The plurality of beads attached to the protein targets can be contained within a 96 well plate such that each well of the 96 well plate contains at least: i) a portion of the biological sample diluted in a suitable buffer, ii) a bead mix, iii) a labeled anti-human secondary antibody, and iv) a portion of the plurality of beads that are each attached to only one type of protein target, and/or epitope binding fragment thereof, of the protein targets, wherein each protein target, and/or epitope binding fragment thereof, is represented in each well of the 96 well plate. The plurality of beads can be magnetic or non-magnetic beads, e.g., polymer or glass beads. The substrate can include a microfluidics device, e.g., a microfluidics device that includes plastic or nitrocellulose. In the methods, measuring the total IgG antibody level in the biological sample can be a normalization control. The detectable label can include labeled anti-human secondary antibody, e.g., an anti-human secondary antibody labeled with a fluorescent label. In the methods, step d) of measuring the levels of the labeled immunocomplexes can include measuring fluorescence. The at least one covariate can be age. The classification algorithm can be a machine learning algorithm.

In embodiments of the methods, the subject has AD-related pathology consistent with pre-symptomatic AD, prodromal AD (mild cognitive impairment), mild-moderate AD, or does not have AD-related pathology. The method can detect the presence of ongoing AD-related pathology with an accuracy of at least 75%. In some embodiments, the subject has PD-related pathology consistent with early-stage PD, or does not have PD-related pathology.

In embodiments of the methods, the method can further include at least one of: conducting at least one additional corroborating diagnostic test of the subject, and treating the subject if the method determines that the subject has AD-related pathology. In some embodiments, the method can further include at least one of: conducting at least one additional corroborating diagnostic test of the subject, and treating the subject if the method determines that the subject has early-stage PD. In such methods, the at least one additional corroborating diagnostic test can be one or more of: neuropsychological evaluation, neuroimaging, evaluation of patient/family history, and cerebrospinal fluid analysis.

In another embodiment, the present disclosure provides a kit for detecting the presence of AD-related pathology in a subject. A kit can include: (a) a protein target set including protein targets MGC31944 or MGC31936, HSH2D, GCDH, CCL19, LGALS1 (Galectin-1), DNAJC8, ICAM-4, and anti-human anti-IgG kappa light chain antibody, and/or epitope binding fragments thereof, wherein the protein targets and/or epitope binding fragments thereof are attached to a substrate including a plurality of beads, each bead attached to only one type of protein target, and/or epitope binding fragment thereof, of the protein targets, (b) a multi-well plate wherein each well of the multi-well plate contains a bead mix and a portion of the plurality of beads that are each attached to only one type of protein target, and/or epitope binding fragment thereof, of the protein targets, wherein each protein target, and/or epitope binding fragment thereof, is represented in each well of the multi-well plate; (c) at least one control; and (d) assay reagents for detection of: i) immunocomplexes formed by binding of each protein target or epitope binding fragment thereof to a corresponding AD-related autoantibody if present in the subject's immunoglobulin-containing biological sample, and ii) immunocomplexes formed by binding of the anti-human anti-IgG kappa light chain antibody to all IgG antibodies having a kappa light chain in the subject's biological sample, wherein the assay reagents include a detectable label for labeling the formed immunocomplexes.

In another embodiment, the present disclosure provides a kit for detecting the presence of PD-related pathology in a subject with early-stage PD. The kit can include: (a) a protein target set including protein targets MARK1, PUSL1, IL20, CCL19, and anti-human anti-IgG kappa light chain antibody, and/or epitope binding fragments thereof, wherein the protein targets and/or epitope binding fragments thereof are attached to a substrate including a plurality of beads, each bead attached to only one type of protein target, and/or epitope binding fragment thereof, of the protein targets, (b) a multi-well plate wherein each well of the multi-well plate contains a bead mix and a portion of the plurality of beads that are each attached to only one type of protein target, and/or epitope binding fragment thereof, of the protein targets, wherein each protein target, and/or epitope binding fragment thereof, is represented in each well of the multi-well plate; (c) at least one control; and (d) assay reagents for detection of: i) immunocomplexes formed by binding of each protein target or epitope binding fragment thereof to a corresponding PD-related autoantibody biomarker if present in the subject's immunoglobulin-containing biological sample, and ii) immunocomplexes formed by binding of the anti-human anti-IgG kappa light chain antibody to all IgG antibodies having a kappa light chain in the subject's biological sample, wherein the assay reagents comprise a detectable label for labeling the formed immunocomplexes.

In another embodiment, the present disclosure provides a kit for detecting the presence of AD-related pathology in a subject. The kit can include: (a) a protein target set including protein targets MGC31944 or MGC31936, HSH2D, GCDH, CCL19, LGALS1 (Galectin-1), DNAJC8, ICAM-4, and anti-human anti-IgG kappa light chain antibody, and/or epitope binding fragments thereof, wherein the protein targets and/or epitope binding fragments thereof are attached to a substrate including a microfluidics device, (b) at least one control; and (c) assay reagents for detection of: i) immunocomplexes formed by binding of each protein target or epitope binding fragment thereof to a corresponding AD autoantibody biomarker if present in the subject's immunoglobulin-containing biological sample, and ii) immunocomplexes formed by binding of the anti-human anti-IgG kappa light chain antibody to all IgG antibodies having a kappa light chain in the subject's biological sample, wherein the assay reagents comprise a detectable label for labeling the formed immunocomplexes.

In another embodiment, the present disclosure provides a kit for detecting the presence of PD-related pathology in a subject with early-stage PD. The kit can include: (a) a protein target set including protein targets MARK1, PUSL1, IL20, CCL19, and anti-human anti-IgG kappa light chain antibody, and/or epitope binding fragments thereof, wherein the protein targets and/or epitope binding fragments thereof are attached to a substrate including a microfluidics device, (b) at least one control; and (c) assay reagents for detection of: i) immunocomplexes formed by binding of each protein target or epitope binding fragment thereof to a corresponding PD-related autoantibody if present in the subject's immunoglobulin-containing biological sample, and ii) immunocomplexes formed by binding of the anti-human anti-IgG kappa light chain antibody to all IgG antibodies having a kappa light chain in the subject's biological sample, wherein the assay reagents comprise a detectable label for labeling the formed immunocomplexes.

In the kits for detecting the presence of AD-related pathology or the presence of PD-related pathology, the detectable label can be labeled anti-human secondary antibody, e.g., an anti-human secondary antibody is labeled with a fluorescent label. The plurality of beads can be magnetic or non-magnetic beads, e.g., polymer or glass beads. The microfluidics device can include plastic or nitrocellulose.

In other embodiments, the present disclosure provides a system for detecting the presence of AD-related pathology in a subject. The system can include: (a) means for receiving an immunoglobulin-containing biological sample from the subject; (b) a protein target set including protein targets MGC31944 or MGC31936, HSH2D, GCDH, CCL19, LGALS1 (Galectin-1), DNAJC8, ICAM-4, and anti-human anti-IgG kappa light chain antibody, and/or epitope binding fragments thereof, (c) a substrate to which the protein targets and/or epitope binding fragments thereof are attached; (d) covariate data from the subject; (e) means for contacting the biological sample with the protein target set, each protein target or epitope binding fragment thereof from the protein target set forms an immunocomplex with a corresponding AD-related autoantibody if present in the biological sample and the recombinant anti-human anti-IgG kappa light chain antibody forms immunocomplexes with all IgG antibodies having a kappa light chain in the biological sample; (f) a detectable label for binding to the immunocomplexes such that the immunocomplexes are labeled; (g) an assay for quantifying the levels of the labeled immunocomplexes to generate levels of AD-related autoantibodies and a total IgG antibody level in the biological sample; and (h) a classification algorithm configured to (1) determine an AD score from the levels of AD-related autoantibodies, total IgG antibody level, and the covariate data, and (2) output the AD score that is compared to a predetermined threshold at or above which there is a likelihood that a subject has ongoing AD-related pathology.

In other embodiments, the present disclosure provides a system for detecting the presence of PD-related pathology in a subject with early-stage PD, the system. The system can include: (a) means for receiving an immunoglobulin-containing biological sample from the subject; (b) a protein target set comprising protein targets MARK1, PUSL1, IL20, CCL19, and anti-human anti-IgG kappa light chain antibody, and/or epitope binding fragments thereof, (c) a substrate to which the protein targets and/or epitope binding fragments thereof are attached; (d) covariate data from the subject; (e) means for contacting the biological sample with the protein target set, each protein target or epitope binding fragment thereof from the protein target set forms an immunocomplex with a corresponding PD-related autoantibody if present in the biological sample and the recombinant anti-human anti-IgG kappa light chain antibody forms immunocomplexes with all IgG antibodies having a kappa light chain in the biological sample; (f) a detectable label for binding to the immunocomplexes such that the immunocomplexes are labeled; and (g) an assay for quantifying the levels of the labeled immunocomplexes to generate levels of PD-related autoantibodies and a total IgG antibody level in the biological sample; and (h) a classification algorithm configured to (1) determine a PD score from the levels of PD-related autoantibodies, total IgG antibody level, and the covariate data, and (2) output the PD score that is compared to a predetermined threshold at or above which there is a likelihood that a subject has early-stage PD-related pathology.

Further described herein is a method of preparing labeled immunocomplexes useful for detecting AD-related pathology. The method can include: (a) collecting an immunoglobulin-containing biological sample from a subject; (b) producing a protein target set including protein targets MGC31944 or MGC31936, HSH2D, GCDH, CCL19, LGALS1 (Galectin-1), DNAJC8, ICAM-4, and anti-human anti-IgG kappa light chain antibody, and/or epitope binding fragments thereof, wherein the protein targets and/or epitope binding fragments thereof are attached to a substrate; (c) preparing labeled immunocomplexes by contacting the biological sample with the protein target set under conditions under which each protein target or epitope binding fragment thereof forms an immunocomplex with a corresponding AD-related autoantibody if present in the biological sample, and the anti-human anti-IgG kappa light chain antibody forms immunocomplexes with all IgG antibodies having a kappa light chain in the biological sample; (d) labeling the immunocomplexes with a detectable label; (e) analyzing the levels of the labeled immunocomplexes to generate levels of AD-related autoantibodies and a total IgG antibody level in the biological sample; (f) obtaining at least one covariate data set generated from the subject; and (g) inputting the levels of the subject's AD-related autoantibodies, total IgG antibody level, and the covariate data into a classification algorithm configured to (1) determine therefrom an AD score, and (2) output the AD score. The method can further include at least one of the following steps: analyzing at least one covariate of the subject to generate covariate data; receiving the AD score; and indicating that the subject has AD-related pathology if the subject's AD score is equal to or greater than a predetermined threshold score at or above which there is a likelihood that a subject has AD-related pathology.

Still further described herein is a method of preparing labeled immunocomplexes useful for detecting PD-related pathology in a subject at early-stage PD. The method can include: (a) collecting an immunoglobulin-containing biological sample from a subject; (b) producing a protein target set including protein targets MARK1, PUSL1, IL20, CCL19, and anti-human anti-IgG kappa light chain antibody, and/or epitope binding fragments thereof, wherein the protein targets and/or epitope binding fragments thereof are attached to a substrate; (c) preparing labeled immunocomplexes by contacting the biological sample with the protein target set under conditions under which each protein target or epitope binding fragment thereof forms an immunocomplex with a corresponding PD-related autoantibody if present in the biological sample, and the anti-human anti-IgG kappa light chain antibody forms immunocomplexes with all IgG antibodies having a kappa light chain in the biological sample; (d) labeling the immunocomplexes with a detectable label; (e) analyzing the levels of the labeled immunocomplexes to generate levels of PD-related autoantibodies and a total IgG antibody level in the biological sample; (f) obtaining at least one covariate data set generated from the subject; and (g) inputting the levels of the subject's PD-related autoantibodies, total IgG antibody level, and the covariate data into a classification algorithm configured to (1) determine therefrom a PD score, and (2) output the PD score. The method can further include at least one of the following steps: analyzing at least one covariate of the subject to generate covariate data; receiving the PD score; and indicating that the subject has ongoing PD-related pathology consistent with early-stage PD if the subject's PD score is equal to or greater than a predetermined threshold score at or above which there is a likelihood that a subject has PD-related pathology consistent with early-stage PD.

In the experiments described below, sera from multiple cohorts, including Alzheimer's Disease Neuroimaging Initiative (ADNI) subjects with pre-symptomatic, prodromal (mild cognitive impairment), and mild-moderate AD, were screened using a bead-based multiplex assay (Luminex xMAP®) technology to predict the probability of the presence of AD-related pathology. A panel of eight protein targets and a covariate were evaluated using randomForest® and Receiver Operating Characteristic (ROC) curves. Use of the protein targets MGC31944 or MGC31936, HSH2D, GCDH, CCL19, LGALS1 (Galectin-1), DNAJC8, and ICAM-4), and anti-human anti-IgG kappa light chain antibody, predicted a patient's probability of an AD diagnosis with 81.0% accuracy and an area under the curve (AUC) of 0.84 (95% CI=0.78-0.91). Inclusion of age as a parameter to the model improved the AUC (0.96; 95% CI=0.93-0.99) and overall accuracy (93.0%). Testing of the panel of eight protein targets resulted in four main findings. First, this panel identified individuals with prodromal AD and mild-moderate AD as positive for AD-related pathology and distinguished them from cognitively normal controls with high overall accuracy. Second, inclusion of age as a covariate significantly improved overall diagnostic performance at all disease stages tested. Third, the panel also achieved detection of AD-related pathology with high overall accuracy in pre-symptomatic AD participants who originally enrolled in ADNI as cognitively normal controls, but a few years later transitioned to prodromal or more advanced AD with confirmed AD pathology. Also in the experiments described below, use of the PD protein targets MARK1, PUSL1, IL20, and CCL19, and anti-human anti-IgG kappa light chain antibody, predicted a patient's probability of a PD diagnosis with an overall accuracy of 87.9%, sensitivity of 94.1%, and specificity of 85.5%. These protein targets also differentiated patients with early-stage PD from those with more advanced (mild-moderate) PD with an accuracy of 97.5%, and could distinguish subjects with early-stage PD from those with other neurological diseases including Alzheimer's disease with an accuracy of 97.0%, and multiple sclerosis with an accuracy of 96.3%, as well as a non-neurological disease, breast cancer, with an accuracy of 97.5%.

### BRIEF DESCRIPTION OF THE DRAWINGS

FIG. 1(a)-FIG. 1(d) are a series of pie charts showing grouping of patient and control sera used in the Training and Testing Sets. FIG. 1(a): Composition of the total participants in the study (n=328). Patients with disease were comprised of 64 pre-symptomatic MCI/AD from ADNI, 71 MCI from ADNI, 24 AD from ADNI, and 33 MCI/AD from MAP. Control patients were comprised of 18 breast cancer, 12 early-stage PD, and 106 non-demented controls from Reprocell. FIG. 1(b) Composition of the Training Set participants in the study (n=184). Patients with disease were comprised of 34 pre-symptomatic MCI/AD from ADNI, 37 MCI from ADNI, 13 AD from ADNI, and 18 MCI/AD from MAP. Control patients were comprised of 18 breast cancer, 12 early-stage PD, and 52 non-demented controls from Reprocell. FIG. 1(c) Composition of the Testing Set participants in the study (n=144). The patients with disease were comprised of 30 pre-symptomatic MCI from ADNI, 34 MCI from ADNI, 11 AD from ADNI, and 15 MCI/AD from MAP. The control patients included 54 non-demented controls from Reprocell. FIG. 1(d) Composition of the Testing Set age-matched participants in the study (n=49). Patients with disease were comprised of 7 pre-symptomatic MCI from ADNI, 11 MCI from ADNI, 2 AD from ADNI, and 4 MCI and 1 AD from MAP. The control patients included 24 non-demented controls from Reprocell.

FIG. 2 is a flow chart of the diagnostic model creation and testing in randomForest®, leading to the final output as the Alzheimer's Disease Probability Score (ADPS).

FIG. 3 is a graph showing a Receiver Operating Characteristic (ROC) curve assessment of autoantibody biomarkers for detection of AD-related pathology in Testing Set subjects; cases (pre-symptomatic, prodromal, and mild-moderate AD) (n=90) vs. cognitively normal controls (n=54) when used alone (green line), with age as an additional parameter (blue line) in a group with non-age-matched controls, and with age as an additional parameter with a more closely age-matched control group (red line). Results show that inclusion of age as an additional parameter significantly increases overall diagnostic accuracy and, thus, the overall utility of the test. The dashed line represents the line of no discrimination. The ROC area under the curve (AUC), sensitivity, specificity, PPV, NPV, and overall accuracy values are shown in Table 5.

FIG. 4 is a histogram showing the distribution of ADPS in Testing Set subjects (n=144) for increasing or decreasing likelihood of the presence of AD-related pathology. Based on a scale of 0-100, a score of 56 or greater indicates a higher likelihood of the presence of AD-related pathology, while a score of 55 or lower indicates a reduced likelihood.

FIG. 5 is a diagram showing the proposed mechanism of disease-associated autoantibodies in the serum and their utility as diagnostic biomarkers of pre-symptomatic disease.

### DETAILED DESCRIPTION OF THE INVENTION

Prior studies of human sera have shown that humans have many thousands of autoantibodies (aABs) in their blood, with evidence supporting the function of this autoantibody system to be clearance of debris from the blood and lymph on a day-to-day basis. Such evidence includes the observations that in overall healthy people, individual autoantibody profiles can be remarkably stable, sometimes over a period of many years, and that certain autoantibodies are selectively increased in the blood in response to the presence of disease and, importantly, these same increases were consistently observed in people with the same disease. The presence of disease triggers consistent disease-associated changes in autoantibody profiles that reflect disease-associated changes in the debris profile exhibited in the blood. In the experiments described herein, the utility of a set of protein targets that specifically bind to a specific panel of AD autoantibody biomarkers was tested for the blood-based detection of AD-related pathology in participants at pre-symptomatic, prodromal, and more advanced stages of AD. The results demonstrate that increased levels of seven specific disease-associated autoantibodies in the blood are useful as biomarkers indicative of the presence of AD-related pathology, identifying not only subjects with prodromal AD or more advanced stages of the disease from normal control subjects, but also individuals at the pre-symptomatic (pre-symptomatic) stage of AD (i.e., cognitively normal individuals without subjective cognitive or memory decline who later transitioned to AD) with high overall accuracy, sensitivity, and specificity. Protein targets MGC31944 or MGC31936, HSH2D, GCDH, CCL19, LGALS1 (Galectin-1), DNAJC8, and ICAM-4 specifically bind to the seven AD-related autoantibodies and thus are used to measure the levels of the seven AD-related autoantibodies. The results of the experiments described below also demonstrate use of the PD protein targets MARK1, PUSL1, IL20, and CCL19, and anti-human anti-IgG kappa light chain antibody, in predicting a patient's probability of a PD diagnosis with high accuracy and differentiating patients with early-stage PD from those with more advanced (mild-moderate) PD with high accuracy and distinguishing subjects with early-stage PD from those with other neurological diseases as well as those with non-neurological disease with high accuracy.

Accordingly, described herein is a method for detecting the presence of pre-symptomatic AD-related pathology in a subject. The method has an overall accuracy of 93%. The method includes obtaining an immunoglobulin-containing biological sample from the subject. The subject is typically a human, but can be any mammal. A subject can have pre-symptomatic AD, prodromal AD (MCI), mild-moderate AD, or may not have AD. Any suitable immunoglobulin-containing biological sample can be obtained and used in the method, e.g., whole blood, serum, cerebrospinal fluid, saliva, and sputum. Immunoglobulin-containing biological samples can be obtained from subjects using any suitable methods. A blood sample may be obtained by methods known in the art including venipuncture or a finger stick. CSF may be obtained by methods known in the art including a lumbar spinal tap. To obtain serum from blood, typically a sample of blood is received and centrifuged at a speed sufficient to pellet all cells and platelets, and the serum to be analyzed is drawn from the resulting supernatant. Sputum and saliva samples may be collected by methods known in the art. The biological samples may be diluted with a suitable buffer.

The method includes providing a protein target set including protein targets MGC31944 or MGC31936, HSH2D, GCDH, CCL19, LGALS1 (Galectin-1), DNAJC8, ICAM-4, and anti-human anti-IgG kappa light chain antibody, and/or epitope binding fragments thereof. Protein targets MGC31944 or MGC31936, HSH2D, GCDH, CCL19, LGALS1 (Galectin-1), DNAJC8, and ICAM-4 bind to their cognate (corresponding) autoantibody biomarkers if present in the biological sample. The anti-human anti-IgG kappa light chain antibody binds indiscriminately to any antibody with a kappa light chain present in the biological sample. This particular combination of autoantibody biomarkers present in the biological sample in combination with the protein target set was used to predict a patient's probability of having ongoing AD-related pathology with 81.0% accuracy and when combined with age as an additional parameter, had an overall accuracy of 93.0%. The sequences of these protein targets used to attract and bind to the autoantibody biomarkers present in the biological fluid are presented in Table 1 below. The protein targets (including epitope binding fragments thereof) can be produced recombinantly (i.e., recombinant protein targets) or synthetically (i.e., synthetic protein targets) by methods known in the art. The protein targets can be produced in vitro using cell-free translation systems and in any of the production methods, purified by any suitable methods. Purification of the protein targets involves tags being attached to the proteins—a standard feature of protein purification. In embodiments, the protein targets are produced in a mammalian, insect or bacterial expression system to ensure correct folding and function. The protein targets as described herein contain some, but not all of the final post-translational modifications that would be found in the naturally occurring versions of these proteins. In all embodiments, the tagged protein targets are not naturally occurring in the body. All of these methods may be automated for high throughput production.

Table 1 below includes the amino acid sequences of the seven protein targets used for detection of autoantibody biomarkers in biological fluids that are indicative of the presence of AD-related pathology described herein. Protein targets MGC31944 and MGC31936 (numbers 1 and (1) in Table 1) are isoforms, and in the methods, systems and kits described herein, typically either MGC31944 or MGC31936 is used. Anti-human anti-IgG Kappa Light Chain antibody is a protein target (used to measure individual serum titers of IgG) as described herein and is included in the AD panel of Table 1.

TABLE 1
AD Panel of 8 Protein Targets and Their Sequences

| No. | Database ID | Protein ID | Protein Name | Amino Acid Sequence |
|---|---|---|---|---|
| 1 | BC022098.1 | AAH22098.1 | cDNA clone | MAWSPLLLTLLAHCTGSWAQSVLT |
| | | | MGC: 31944 | QPPSVSGAPGQRVTISCTGSSSNIGA |
| | | | IMAGE: 4878869 | GYDVHWYQQLPGTAPKLLIY |
| | | | | GNSNRPSGVPDRFSGSKSGTSASLAI |
| | | | | TGLQAEDEADYYCQSYDYSLSASGV |
| | | | | FGGGTKLTVLGQPKAAPSV |
| | | | | TLFPPSSEELQANKATLVCLISDFYP |
| | | | | GAVTVAWKADSSPVKAGVETTTPSK |
| | | | | QSNNKYAASSYLSLTPEQW |
| | | | | KSHRSYSCQVTHEGSTVEKTVAPTE |
| | | | | CS (SEQ ID NO: 1) |
| (1) | BC020233.1 | AAH20233.1 | cDNA clone | MAWSPLLLTLLAHCTGSWAQSVLT |
| | | | MGC: 31936 | QPPSVSGAPGQRVTISCTGSSSNIGA |
| | | | IMAGE: 4765518 | GYDVHWYQQLPGTAPKLLIY |
| | | | | GNSNRPSGVPDRFSGSKSGTSASLAI |
| | | | | TGLQAEDEADYYCQSYDSSLSGFVV |
| | | | | FGGGTKLTVLGQPKAAPSV |
| | | | | TLFPPSSEELQANKATLVCLISDFYP |
| | | | | GAVTVAWKADSSPVKAGVETTTPSK |
| | | | | QSNNKYAASSYLSLTPEQW |
| | | | | KSHRSYSCQVTHEGSTVEKTVAPTE |
| | | | | CS (SEQ ID NO: 2) |
| 2 | NM_032855.4 | NP_116244.1 | hematopoietic | MTEAGKLPLPLPPRLDWFVHTQMG |
| | | | SH2 domain | QLAQDGVPEWFHGAISREDAENLLE |
| | | | containing | SQPLGSFLIRVSHSHVGYTLS |
| | | | (HSH2D) | YKAQSSCCHFMVKLLDDGTFMIPGE |
| | | | | KVAHTSLDALVTFHQQKPIEPRREL |
| | | | | LTQPCRQKDPANVDYEDLFL |
| | | | | YSNAVAEEAACPVSAPEEASPKPVLC |
| | | | | HQSKERKPSAEMNRITTKEATSSCPP |
| | | | | KSPLGETRQKLWRSLKML |
| | | | | PERGQRVRQQLKSHLATVNLSSLLD |
| | | | | VRRSTVISGPGTGKGSQDHSGDPTS |
| | | | | GDRGYTDPCVATSLKSPSQP |
| | | | | QAPKDRKVPTRKAERSVSCIEVTPG |
| | | | | DRSWHQMVVRALSSQESKPEHQGL |
| | | | | AEPENDQLPEEYQQPPPFAPG |
| | | | | YC (SEQ ID NO: 3) |
| 3 | OLD: | NP_006265.1 | chemokine (C-C | MALLLALSLLVLWTSPAPTLSGTND |
| | PHC1244 | | motif) ligand 19 | AEDCCLSVTQKPIPGYIVRNFHYLLI |
| | New: | | (CCL19) | KDGCRVPAVVFTTLRGRQL |
| | NM_006274.3 | | | CAPPDQPWVERIIQRLQRTSAKMKR |
| | | | | RSS (SEQ ID NO: 4) |
| 4 | NM_000159.4 | NP_000150.1 | glutaryl- | MALRGVSVRLLSRGPGLHVLRTWV |
| | | | Coenzyme A | SSAAQTEKGGRTQSQLAKSSRPEFD |
| | | | dehydrogenase | WQDPLVLEEQLTTDEILIRDT |
| | | | (GCDH), nuclear | FRTYCQERLMPRILLANRNEVFHRE |
| | | | gene encoding | IISEMGELGVLGPTIKGYGCAGVSSV |
| | | | mitochondrial | AYGLLARELERVDSGYRSA |
| | | | protein, | MSVQSSLVMHPIYAYGSEEQRQKYL |
| | | | transcript | PQLAKGELLGCFGLTEPNSGSDPSS |
| | | | variant 1 | METRAHYNSSNKSYTLNGTK |
| | | | | TWITNSPMADLFVVWARCEDGCIRG |
| | | | | FLLEKGMRGLSAPRIQGKFSLRASA |
| | | | | TGMIIMDGVEVPEENVLPGA |
| | | | | SSLGGPFGCLNNARYGIAWGVLGAS |
| | | | | EFCLHTARQYALDRMQFGVPLARN |
| | | | | QLIQKKLADMLTEITLGLHACLQLG |
| | | | | RLKDQDKAAPEMVSLLKRNNCGKA |
| | | | | LDIARQARDMLGGNGISDEYHVIRH |
| | | | | AMNLEAVNTYEGTHDIHALILGRAI |
| | | | | TGIQAFTASK (SEQ ID NO: 5) |
| 5 | NM_002305.4 | NP_002296.1 | lectin, | MACGLVASNLNLKPGECLRVRGEV |
| | | | galactoside- | APDAKSFVLNLGKDSNNLCLHFNPR |
| | | | binding, soluble, | FNAHGDANTIVCNSKDGGAWGTEQ |
| | | | 1 (galectin 1) | REAVFPFQPGSVAEVCITFDQANLTV |
| | | | (LGALS1) | KLPDGYEFKFPNRLNLEAINYMAAD |
| | | | | GDFKIKCVAFD (SEQ ID NO: 6) |
| 6 | NM_014280.3 | NP_055095.2 | DnaJ homolog | MAASGESGTSGGGGSTEEAFMTFYS |
| | | | subfamily C | EVKQIEKRDSVLTSKNQIERLTRPGS |
| | | | member 8 | SYFNLNPFEVLQIDPEVTD |
| | | | | EEIKKRFRQLSILVHPDKNQDDADR |
| | | | | AQKAFEAVDKAYKLLLDQEQKKRA |
| | | | | LDVIQAGKEYVEHTVKERKKQ |
| | | | | LKKEGKPTIVEEDDPELFKQAVYKQ |
| | | | | TMKLFAELEIKRKEREAKEMHERK |
| | | | | RQREEEIEAQEKAKREREWQK |
| | | | | NFEESRDGRVDSWRNFQANTKGKK |
| | | | | EKKNRTFLRPPKVKMEQRE (SEQ ID |
| | | | | NO: 7) |
| 7 | NM_001544.5 | NP_001535.1 | intercellular | MGSLFPLSLLFFLAAAYPGVGSALG |
| | | | adhesion | RRTKRAQSPKGSPLAPSGTSVPFWV |
| | | | molecule 4 | RMSPEFVAVQPGKSVQLNCSNSCPQ |
| | | | (Landsteiner- | PQNSSLRTPLRQGKTLRGPGWVSYQ |
| | | | Wiener blood | LLDVRAWSSLAHCLVTCAGKTRWA |
| | | | group) (ICAM4), | TSRITAYKPPHSVILEPPVLKGRKYT |
| | | | transcript | LRCHVTQVFPVGYLVVTLRHGSRVI |
| | | | variant 1 | YSESLERFTGLDLANVTLTYEFAAGP |
| | | | | RDFWQPVICHARLNLDGLVVRNSSA |
| | | | | PITLMLAWSPAPTALASGSIAALVGI |
| | | | | LLTVGAAYLCKCLAMKSQA (SEQ ID |
| | | | | NO: 8) |
| 8 | Anti-human | | | No sequence is provided here |
| | anti-IgG | | | as any commercially available |
| | Kappa Light | | | anti-kappa antibody can be used |
| | Chain | | | |
| | antibody | | | |

Also described herein is a method for detecting the presence of early PD-related pathology in a subject. The method includes providing a protein target set including anti-human anti-IgG kappa light chain antibody and 4 PD-related protein targets: 1) Serine/threonine-protein kinase (MARK1); 2) tRNA pseudouridine synthase-like 1 (PUSL1); 3) Interleukin-20 (IL20); and 4) C—C motif chemokine 19 (CCL19), and/or epitope binding fragments thereof. Table 2 below includes the amino acid sequences of the 4 PD-related protein targets (included in a panel or set with anti-human anti-IgG kappa light chain antibody) that bind to their cognate antibody biomarkers present in the biological fluid described herein.

TABLE 2
Panel of 4 PD-related Protein Targets and Their Sequences

| Number (in | | | | |
|---|---|---|---|---|
| order of | Database | Protein | | |
| importance) | ID | Name | Protein Name | Amino Acid Sequence |
| 1 | PV4395 | NP_061120.3 | Serine/threonine- | MSARTPLPTVNERDTENHTS |
| | New: | | protein kinase | VDGYTEPHIQPTKSSSRQNIP |
| | NM_018650.5 | | (MARK1) | RCRNSITSATDEQPHIGNYRL |
| | | | | QKTIGKGNFAKVKLARHVL |
| | | | | TGREVAVKIIDKTQLNPTSL |
| | | | | QKLFREVRIMKILNHPNIVK |
| | | | | LFEVIETEKTLYLVMEYASG |
| | | | | GEVFDYLVAHGRMKEKEAR |
| | | | | AKFRQIVSAVQYCHQKYIVH |
| | | | | RDLKAENLLLDGDMNIKIAD |
| | | | | FGFSNEFTVGNKLDTFCGSP |
| | | | | PYAAPELFQGKKYDGPEVD |
| | | | | VWSLGVILYTLVSGSLPFDG |
| | | | | QNLKELRERVLRGKYRIPFY |
| | | | | MSTDCENLLKKLLVLNPIKR |
| | | | | GSLEQIMKDRWMNVGHEEE |
| | | | | ELKPYTEPDPDFNDTKRIDI |
| | | | | MVTMGFARDEIN |
| | | | | DALINQKYDEVMATYILLGR |
| | | | | KPPEFEGGESLSSGNLCQRS |
| | | | | RPSSDLNNSTLQSPAHLKVQ |
| | | | | RSISANQKQRRFSDHAGPSIP |
| | | | | PAVSYTKRPQANSVESEQKE |
| | | | | EWDKDVARKLGSTTVGSKS |
| | | | | EMTASPLVGPERKKSSTIPSN |
| | | | | NVYSGGSMARRNTYVCERT |
| | | | | TDRYVALQNGKDSSLTEMS |
| | | | | VSSISSAGSSVASAVPSARPR |
| | | | | HQKSMSTSGHPIKVTLPTIK |
| | | | | DGSEAYRPGTTQRVPAASPS |
| | | | | AHSISTATPDRTRFPRGSSSR |
| | | | | STFHGEQLRERRSVAYNGPP |
| | | | | ASPSHETGAFAHARRGTSTG |
| | | | | IISKITSKFVRRDPSEGEASG |
| | | | | RTDTSRSTSGEPKERDKEEG |
| | | | | KDSKPRSLRFTWSMKTTSS |
| | | | | MDPNDMMREIRKVLDANNC |
| | | | | DYEQKERFLLFCVHGDARQ |
| | | | | DSLVQWEMEVCKLPRLSLN |
| | | | | GVRFKRISGTSIAFKNIASKI |
| | | | | ANELKL (SEQ ID NO: 9) |
| 2 | NM_153339.3 | NP_699170.1 | tRNA pseudouridine | MSSAPASGSVRARYLVYFQY |
| | | | synthase-like 1 | VGTDENGVAAVRGTQRAVG |
| | | | (PUSL1) | VQNYLEEAAERLNSVEPVRF |
| | | | | TISSRTDAGVHALSNAAHLD |
| | | | | VQRRSGRPPFPPEVLAEALN |
| | | | | THLRHPAIRVLRAFRVPSDF |
| | | | | HARHAATSRTYLYRLATGC |
| | | | | HR |
| | | | | RDELPVFERNLCWTLPADCL |
| | | | | DMVAMQEAAQHLLGTHDFS |
| | | | | AFQSAGSPVPSPVRTLRRVS |
| | | | | VSPGQASPLVTPEESRKLRF |
| | | | | WNLEFESQSFLYRQVRRMT |
| | | | | AVLVAVGLGALAPAQVKTIL |
| | | | | ESQDPLGKHQTRVAPAHGL |
| | | | | FLK |
| | | | | SVLYGNLGAASCTLQGPQFG |
| | | | | SHG (SEQ ID NO: 10) |
| 3 | PHC0205 | NP_061194.2 | Interleukin-20 | MKASSLAFSLLSAAFYLLWT |
| | New: | | (IL20) | PSTGLKTLNLGSCVIATNLQ |
| | NM_018724.4 | | | EIRNGFSEIRGSVQAKDGNID |
| | | | | IRILRRTESLQDTKPANRCCL |
| | | | | LRHLLRLYLDRVFKNYQTP |
| | | | | DHYTLRKISSLANSFLTIKKD |
| | | | | LRLCHAHMTCHCGEEAMK |
| | | | | KYSQILSHFEKLEPQAAVVK |
| | | | | ALGELDILLQWMEETE (SEQ |
| | | | | ID NO: 11) |
| 4 | PHC1244 | NP_006265.1 | C-C motif | MALLLALSLLVLWTSPAPTL |
| | New: | | chemokine 19 | SGTNDAEDCCLSVTQKPIPG |
| | NM_006274.3 | | (CCL19) | YIVRNFHYLLIKDGCRVPAV |
| | | | | VFTTLRGRQLCAPPDQPWV |
| | | | | ERIIQRLQRTSAKMKRRSS |
| | | | | (SEQ ID NO: 12) |

The protein targets are attached or coupled to a substrate by any suitable means. In the experiments described below, the protein targets were attached (coupled) to the substrate by covalently coupling. Additional ways to attach protein targets include use of a linker. The substrate can be any suitable solid phase for directly or indirectly attaching the protein targets. Examples of substrates include microtiter or microassay plate, slide, magnetic bead, non-magnetic bead, column, matrix, membrane, or sheet, and may be composed of a synthetic material such as polystyrene, polyvinyl chloride, polyamide, or other synthetic polymers, natural polymers such as cellulose, derivatized natural polymers such as cellulose acetate or nitrocellulose, and glass, for example glass fibers. Typically, a plurality of individually addressable protein targets are immobilized on the surface of the substrate, e.g., a single type of protein target or epitope binding fragment thereof attached to an individual bead. In these embodiments, the individually addressable protein targets are attached to a plurality of beads (e.g., Luminex beads) such that each bead is attached to only one type of protein target and/or an epitope binding fragment thereof. The plurality of beads can be magnetic or non-magnetic beads. The plurality of beads can be, as examples, polymer or glass beads. The beads can be coded such that beads coupled to each of the distinct protein targets can be discriminated from one another. In such embodiments, attaching or coupling the protein targets to the beads can be done using any suitable method. For example, coupling of the protein targets to the beads can be carried out using commercially available kits such as the Luminex xMAP Antibody Coupling Kit according to manufacturer's instructions. Similar coupling chemistry can be used for preparation or manufacturing of protein target sets attached to substrates in scaled up methods. Methods of attaching proteins and peptides to beads for use in a bead-based assay (e.g., a bead-based diagnostic assay) are well known in the art, and described in, e.g., U.S. Pat. Nos. 8,946,393 and 10,859,574, incorporated herein by reference in their entireties. In embodiments, the plurality of beads attached to the protein targets are contained within a multi-well plate, e.g., a 96 well plate such that each well of the 96 well plate contains at least: i) a portion of the biological sample diluted in a suitable buffer, ii) a bead mix, iii) a labeled anti-human secondary antibody, and iv) a portion of the plurality of beads that are each attached to only one type of protein target, and/or epitope binding fragment thereof, wherein each protein target, and/or epitope binding fragment thereof, is represented in each well of the 96 well plate. This embodiment is described further in the Examples below.

In some embodiments, the individually addressable protein targets are attached to a microfluidics device. In embodiments, the microfluidics device is constructed of plastic or nitrocellulose. Methods of disease diagnosis using microfluidics devices are well known in the art and described in, e.g., U.S. Pat. Nos. 11,345,947, and 11,287,432, incorporated herein by reference in their entireties. In some embodiments, the individually addressable protein targets are immobilized on the surface to form an array. Methods of disease diagnosis using arrays are well known in the art and described in, e.g., U.S. Pat. Nos. 10,132,817 and 9,664,687, and U.S. application Ser. No. 16/061,534, all incorporated herein by reference in their entireties. The substrates may be used in suitable shapes, such as films, sheets, or plates, or may be coated onto or bonded or laminated to appropriate inert carriers, such as paper, glass, plastic films, or fabrics.

In the method, the protein target set is contacted with the biological sample under conditions under which each of MGC31944 or MGC31936, HSH2D, GCDH, CCL19, LGALS1 (Galectin-1), DNAJC8, and ICAM-4, or epitope binding fragments thereof, forms an immunocomplex with a corresponding AD-related autoantibody if present in the biological sample, and the anti-human anti-IgG kappa light chain antibody forms immunocomplexes with all IgG antibodies having a kappa light chain in the biological sample. For example, if the biological sample contains AD-related autoantibodies against HSH2D (or epitope binding fragment thereof), in the method, the AD-related autoantibodies against HSH2D will form immunocomplexes with the protein target HSH2D (or epitope binding fragment thereof). Typical conditions under which immunocomplexes are formed include the protein target is present and folded in the correct manner, the subject's biological sample (e.g., sera) contains an AD-related autoantibody which recognizes a protein target, and there is sufficient time and energy (e.g., shaking), and appropriate pH and temperature for the complexes to form. In the method, because anti-kappa IgG antibodies account for approximately ⅔ of total IgG antibodies in a biological sample, the protein target anti-human anti-IgG kappa light chain antibody provides an estimate of the IgG antibody concentration in a biological sample. The anti-human anti-IgG kappa light chain antibody is thus useful for accounting for individual differences in total IgG levels, allowing direct comparison of serum samples from different individuals. Normal IgG ranges in adults are as follows: IgG 6.0-16.0 g/L; IgA 0.8-3.0 g/L; IgM 0.4-2.5 g/L. For instance, if a patient's biological sample has what is considered a low anti-IgG kappa light chain antibody value (e.g., an anti-Kappa signal/Background ratio of less than 1.2 in some embodiments), that biological sample may be discarded (and another biological sample obtained from the patient). Thus, the anti-IgG kappa light chain antibody value that is measured acts as a quality control measure (a normalization control) of a patient's biological sample. In the protein target set, protein target MGC31944 is an anti-lambda IgG antibody. In embodiments of the method in which each protein target is processed (contacted with biological sample) in the same space (e.g., a well in a 96 well plate as described in the experiments described below), so as not to interfere with protein target MGC31944, an anti-kappa antibody was chosen to measure IgG kappa antibody levels in a sample. A typical method as described herein includes measuring human AD-related autoantibody levels, and in order to normalize the sample levels the following strategy was used and described in the Examples below: a recombinant rabbit anti-human kappa light chain antibody was coupled to Luminex xMAP microspheres and was used as a capture antibody for human IgG.

In the method, a detectable label is added to the immunocomplexes such that the immunocomplexes are labeled, and the levels of the labeled immunocomplexes are measured to generate levels of AD-related autoantibodies and a total IgG antibody level in the biological sample. Any suitable detectable label can be used. For example, a secondary antibody that is coupled to an indicator reagent comprising a signal generating compound. The secondary antibody may be an anti-human IgG or IgM antibody. Indicator reagents include chromogenic agents, catalysts such as enzyme conjugates, fluorescent compounds such as Phycoerythrin (PE), fluorescein, rhodamine and AlexaFluor, chemiluminescent compounds such as dioxetanes, acridiniums, phenanthridiniums, ruthenium, and luminol, radioactive elements, direct visual labels, as well as cofactors, inhibitors and magnetic particles. Examples of enzyme conjugates include alkaline phosphatase, horseradish peroxidase and beta-galactosidase. The levels of the labeled immunocomplexes can be measured by any suitable method. In some embodiments, measuring the levels of the labeled immunocomplexes includes measuring fluorescence. For example, the detectable label can be anti-human secondary antibody labeled with Phycoerythrin and the fluorescence is measured using an instrument that measures fluorescence.

In the method, at least one covariate data set generated from the subject is obtained. A non-limiting list of covariates that can be used in the methods includes age, cognitive complaint, Clinical Dementia Rating (CDR) score, Mini Mental State Examination (MMSE), CSF amyloid-beta₁₋₄₂, CSF tau, and CSF ptau levels. There is no upper limit for the number of covariates that can be analyzed. In the experiments described below, age was used as a covariate. In the method, determining an AD score for the subject is achieved by A) inputting the covariate data, levels of the subject's AD-related autoantibodies, and total IgG antibody level into a classification algorithm. The classification algorithm is configured to determine therefrom an AD score and output the AD score. In a typical embodiment, the classification algorithm is a machine learning algorithm such as randomForest®. Methods of randomForest® analysis of monitoring and diagnosis of disease are known in the art and described in U.S. patent application Ser. Nos. 17/708,328 and 17/553,498, incorporated herein by reference in their entireties. Any suitable machine learning algorithm, however, can be used, e.g., Linear regression, Logistic regression, Decision tree, SVM algorithm, Naïve Bayes algorithm, KNN algorithm, K-means, randomForest® algorithm, Dimensionality reduction algorithms, Gradient boosting algorithm and AdaBoosting algorithm.

The method can further include analyzing at least one (e.g., 1, 2, 3, 4, 5, 10, 15) covariate of the subject to generate covariate data, receiving the AD score, and determining that the subject has ongoing AD-related pathology if the subject's AD score is equal to or greater than a predetermined threshold score at or above which there is a likelihood that a subject has AD-related pathology. The AD score is generated as a ratio, and typically reported to the patient as a percent of the ratio. In a typical method, the covariate data, levels of the subject's AD-related autoantibodies, and total IgG antibody level are raw values that are normalized as ratio values by dividing each raw value by the background value for each of the 8 protein targets, i.e., signal over background. These ratio values are then analyzed by randomForest® which is a type of machine learning, that creates a classification algorithm using many uncorrelated decision trees (a forest) that function together as an ensemble to generate a class prediction, in the experiments described below, either Case (AD) or Control. In other words, randomForest® creates the algorithm used to analyze the Testing Set data. The class with the most votes is the model prediction for each sample. In this example there are no single values as cutoffs for each individual marker, rather, it is the decision of the panel as a whole. Additionally, the covariate of age functions as a continuous variable in the randomForest® decision. The information for this covariate is processed by randomForest® in the class prediction at the same time as the values of the panel of protein targets. The predetermined threshold score typically is a single threshold value for the prediction, where if a patient (subject) fell above the threshold, that patient would be classified as a case, and if the patient fell below the threshold, the patient would be classified as a control. In addition to the randomForest® prediction of just case or control, randomForest® provides a numerical probability ratio output e.g., 0.993 AD and 0.007 CON so therefore this patient would be classified as having AD-related pathology. The probability ratio is one of the outputs after all the input singles have been analyzed. The anti-kappa signal is one of the input signals.

The method can include one or more controls in addition to the normalization control provided by measuring the total IgG antibody level. Examples of such additional controls include buffer (for background), antigen-specific antibodies, tag-specific antibodies, and sera (Control, Pooled, Synthetic).

The method can further include at least one of: conducting at least one (e.g., 1, 2, 3, 4, 5, etc.) additional corroborating diagnostic test of the subject, and treating the subject if the method determined that the subject has ongoing AD-related pathology. The at least one additional corroborating diagnostic test can be, as examples, neuropsychological evaluation, neuroimaging, evaluation of patient/family history, and CSF analysis. Traditional methods to diagnose AD most often involve a clinical judgement made by weighing data derived from some combination of patient history, a wide variety of simple and more extensive neuropsychological screeners and tests, diagnostic imaging, and CSF analysis of various biomarkers, such as Aβ42 and Aβ40, total Tau, and various forms of phosphorylated Tau (pTau).

In another embodiment, described herein are kits for detecting the presence of AD-related pathology in a subject. In embodiments, a kit includes: a protein target set including protein targets MGC31944 or MGC31936, HSH2D, GCDH, CCL19, LGALS1 (Galectin-1), DNAJC8, ICAM-4, and anti-human anti-IgG kappa light chain antibody, and/or epitope binding fragments thereof, the protein targets and/or epitope binding fragments thereof being attached to a substrate including a plurality of beads, each bead attached to only one type of protein target, and/or epitope binding fragment thereof, of the protein targets; a multi-well plate (e.g., 96 well plate) wherein each well of the multi-well plate contains a bead mix and a portion of the plurality of beads that are each attached to only one type of protein target, and/or epitope binding fragment thereof, of the protein targets, such that each protein target, and/or epitope binding fragment thereof, is represented in each well of the multi-well plate; at least one (e.g., 1, 2, 3, 4, 5, etc.) control; and assay reagents. In such embodiments, the plurality of beads are magnetic or non-magnetic beads, e.g., polymer or glass beads. In other embodiments, the kit includes a protein target set including protein targets MGC31944 or MGC31936, HSH2D, GCDH, CCL19, LGALS1 (Galectin-1), DNAJC8, ICAM-4, and anti-human anti-IgG kappa light chain antibody, and/or epitope binding fragments thereof, the protein targets and/or epitope binding fragments thereof being attached to a substrate including a microfluidics device. The microfluidics device can be made of, as examples, plastic or nitrocellulose.

In the kits, the assay reagents are for detection of: i) immunocomplexes formed by binding of each protein target or epitope binding fragment thereof to a corresponding AD-related autoantibody biomarker if present in the subject's immunoglobulin-containing biological sample, and ii) immunocomplexes formed by binding of the anti-human anti-IgG kappa light chain antibody to all IgG antibodies having a kappa light chain in the subject's biological sample. In such embodiments, the microfluidics device can include plastic or nitrocellulose. In a typical kit, the assay reagents include a detectable label for labeling the formed immunocomplexes. The detectable label can be, for example, anti-human secondary antibody that is labeled with a fluorescent label (e.g., Phycoerythrin). In the kits, the at least one control can include one or more of buffer (for background), antigen-specific antibodies, tag-specific antibodies, and sera (Control, Pooled, Synthetic). Kits also typically include a container and packaging. Instructional materials for preparation and use of the protein target set (protein antigen targets) described herein are generally included. While the instructional materials typically include written or printed materials, they are not limited to such. Any medium capable of storing such instructions and communicating them to an end user is encompassed by the kits herein. Such media include, but are not limited to electronic storage media, optical media, and the like. Such media may include addresses to internet sites that provide such instructional materials.

Also described herein is a system for detecting the presence of AD-related pathology in a subject. The system includes (a) means for receiving an immunoglobulin-containing biological sample from the subject; (b) a protein target set including protein targets MGC31944 or MGC31936, HSH2D, GCDH, CCL19, LGALS1 (Galectin-1), DNAJC8, ICAM-4, and anti-human anti-IgG kappa light chain antibody, and/or epitope binding fragments thereof, (c) a substrate to which the protein targets and/or epitope binding fragments thereof are attached; (d) covariate data from the subject; (e) means for contacting the biological sample with the protein target set, each protein target or epitope binding fragment thereof from the protein target set forms an immunocomplex with a corresponding AD-related autoantibody if present in the biological sample and the recombinant anti-human anti-IgG kappa light chain antibody forms immunocomplexes with all IgG antibodies having a kappa light chain in the biological sample; (f) a detectable label for binding to the immunocomplexes such that the immunocomplexes are labeled; and (g) an assay for quantifying the levels of the labeled immunocomplexes to generate levels of AD-related autoantibodies and a total IgG antibody level in the biological sample; and (h) a classification algorithm configured to (1) determine an AD score from the levels of AD-related autoantibodies, total IgG antibody level, and the covariate data, and (2) output the AD score that is compared to a predetermined threshold at or above which there is a likelihood that a subject has ongoing AD-related pathology. In the system, the immunoglobulin-containing biological sample, protein target set, substrate, covariate data, conditions, detectable label, measurements, classification algorithm, are as described above (and below) for the methods of detecting the presence of AD-related pathology in a subject.

A system for detecting the presence of PD-related pathology in a subject with early-stage PD is also described herein. Such a system can include: (a) means for receiving an immunoglobulin-containing biological sample from the subject; (b) a protein target set comprising protein targets MARK1, PUSL1, IL20, CCL19, and anti-human anti-IgG kappa light chain antibody, and/or epitope binding fragments thereof, (c) a substrate to which the protein targets and/or epitope binding fragments thereof are attached; (d) covariate data from the subject; (e) means for contacting the biological sample with the protein target set, each protein target or epitope binding fragment thereof from the protein target set forms an immunocomplex with a corresponding PD-related autoantibody if present in the biological sample and the recombinant anti-human anti-IgG kappa light chain antibody forms immunocomplexes with all IgG antibodies having a kappa light chain in the biological sample; (f) a detectable label for binding to the immunocomplexes such that the immunocomplexes are labeled; (g) an assay for quantifying the levels of the labeled immunocomplexes to generate levels of PD-related autoantibodies and a total IgG antibody level in the biological sample; and (h) a classification algorithm configured to (1) determine a PD score from the levels of PD-related autoantibodies, total IgG antibody level, and the covariate data, and (2) output the PD score that is compared to a predetermined threshold at or above which there is a likelihood that a subject has early-stage PD-related pathology.

Further described herein is a method of preparing labeled immunocomplexes useful for detecting AD-related pathology. The method includes (a) collecting an immunoglobulin-containing biological sample from a subject; (b) producing a protein target set including protein targets MGC31944 or MGC31936, HSH2D, GCDH, CCL19, LGALS1 (Galectin-1), DNAJC8, ICAM-4, and anti-human anti-IgG kappa light chain antibody, and/or epitope binding fragments thereof, wherein the protein targets and/or epitope binding fragments thereof are attached to a substrate; (c) preparing labeled immunocomplexes by contacting the biological sample with the protein target set under conditions under which each protein antigen target or epitope binding fragment thereof forms an immunocomplex with a corresponding AD-related autoantibody if present in the biological sample, and the anti-human anti-IgG kappa light chain antibody forms immunocomplexes with all IgG antibodies having a kappa light chain in the biological sample; (d) labeling the immunocomplexes with a detectable label; (e) analyzing the levels of the labeled immunocomplexes to generate levels of AD-related autoantibodies and a total IgG antibody level in the biological sample; (f) obtaining at least one covariate data set generated from the subject; and (g) inputting the levels of the subject's AD-related autoantibodies, total IgG antibody level, and the covariate data into a classification algorithm configured to (1) determine therefrom an AD score, and (2) output the AD score. The method can further include one or more of: analyzing at least one covariate of the subject to generate covariate data; receiving the AD score; and indicating that the subject has AD-related pathology if the subject's AD score is equal to or greater than a predetermined threshold score at or above which there is a likelihood that a subject has AD-related pathology. In the method, the collection of immunoglobulin-containing biological sample, production of protein target set, preparation of labeled immunocomplexes, labeling of the immunocomplexes, analysis of the levels of the labeled immunocomplexes and of the at least one covariate (e.g., age), inputting of the levels of the subject's AD-related autoantibodies, total IgG antibody level, and the covariate data into a classification algorithm, receipt of the AD score, and indication as to whether or not the subject has ongoing AD-related pathology are performed as described above (and below) for the methods of diagnosing (detecting) AD in a subject.

A method of preparing labeled immunocomplexes useful for detecting PD-related pathology in a subject at early-stage PD is also described here. The method can include: (a) collecting an immunoglobulin-containing biological sample from a subject; (b) producing a protein target set comprising protein targets MARK1, PUSL1, IL20, CCL19, and anti-human anti-IgG kappa light chain antibody, and/or epitope binding fragments thereof, wherein the protein targets and/or epitope binding fragments thereof are attached to a substrate; (c) preparing labeled immunocomplexes by contacting the biological sample with the protein target set under conditions under which each protein target or epitope binding fragment thereof forms an immunocomplex with a corresponding PD-related autoantibody if present in the biological sample, and the anti-human anti-IgG kappa light chain antibody forms immunocomplexes with all IgG antibodies having a kappa light chain in the biological sample; (d) labeling the immunocomplexes with a detectable label; (e) analyzing the levels of the labeled immunocomplexes to generate levels of PD-related autoantibodies and a total IgG antibody level in the biological sample; (f) obtaining at least one covariate data set generated from the subject; and (g) inputting the levels of the subject's PD-related autoantibodies, total IgG antibody level, and the covariate data into a classification algorithm configured to (1) determine therefrom a PD score, and (2) output the PD score. In the method, the collection of immunoglobulin-containing biological sample, production of protein target set, preparation of labeled immunocomplexes, labeling of the immunocomplexes, analysis of the levels of the labeled immunocomplexes and of the at least one covariate (e.g., age), inputting of the levels of the subject's PD-related autoantibodies, total IgG antibody level, and the covariate data into a classification algorithm, receipt of the PD score, and indication as to whether or not the subject has ongoing PD-related pathology consistent with early-stage PD are performed as described above (and below) for the methods of diagnosing (detecting) PD in a subject.

In the experiments described below, a Luminex magnetic bead-based analytical platform was used. However, the detection of serum autoantibody biomarkers with the protein targets described herein is not platform-specific. Any suitable technology platform or device can be used. A non-limiting list of examples of suitable technology platforms includes enzyme linked immunosorbent assay (ELISA), clinical chemistry, immunofluorescence, enzyme immunoassay, indirect fluorescence antibody assays, immunodiagnostics, and peptide binding. Another example of a suitable technology platform is a closed system, in which software/and or equipment is licensed from a copyright holder. Examples of such closed systems include: Cobas® systems by Roche Diagnostics Corporation (Indianapolis, IN); BioPlex, PhD Ix, Genius and Evolis Systems by Bio-Rad (Hercules, California); AU, DxC, UniCel Dxl, Access (Immunoassay) and Immage® Series Systems by Beckman Coulter (Brea, California); Accelerator, Alinity, Aliniq, Architect, Cell-Dyn and GLP systems by Abbott Core Laboratory (Abbott Park, Illinois); ThunderBold® Systems by Gold Standard Diagnostics (Davis, CA); and VITROS® Series Systems by Ortho Clinical Diagnostics (Davis, CA).

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

“Alzheimer's Disease (AD)” is a clinical and pathological continuum that includes pre-clinical (pre-symptomatic) AD, prodromal AD (mild cognitive impairment due to the presence of ongoing early AD pathology) and more advanced AD dementia stages. The term “early-stage AD” is used herein to refer to pre-clinical and prodromal AD, the two stages recognized before the AD dementia stage where the profile of clinical symptoms is recognizable as characteristic of AD dementia. “Prodromal AD” is considered an early-stage of AD pathology where patients display some symptoms such as cognitive and memory complaints that have not yet reached the level where they can be collectively characterized as bona fide AD dementia. “Prodromal AD” is synonymous with “mild-cognitive impairment (MCI) due to ongoing AD-related pathology. The AD continuum also includes mild-moderate AD and full AD dementia. The methods and kits described herein typically detect the presence of AD-related pathology in individuals at pre-symptomatic, prodromal, and mild-moderate AD stages.

As used herein, “more advanced stages of AD” typically refers to individuals who have a confirmed diagnosis of AD.

AD-related pathology includes the hallmark features of neuronal loss, synaptic loss, amyloid plaques formation, neurofibrillary tangles, and associated local inflammatory changes.

The terms “Parkinson's Disease” and “PD” mean a chronic and progressive disorder of the central nervous system involving neuronal and synaptic loss, and resulting in motor impairment. It is the second most common neurodegenerative disease in the world, after Alzheimer's disease. Early-stage PD is considered a score of 2 or less on the Hoehn and Yahr rating scale. PD-related pathology includes the hallmark features of neuronal loss, synaptic loss, alpha-synuclein deposition (lewy bodies), and associated local inflammatory changes.

The terms “specific binding” and “specifically binds” refer to that binding which occurs between such paired species as enzyme/substrate, receptor/agonist, antibody/antigen, etc., and which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs.

As used herein the term “immunoglobulin-containing biological sample” means a biological sample (e.g., from a human) that contains immunoglobulins, i.e., any of a class of proteins which function as antibodies, including AD-related autoantibodies and PD-related autoantibodies.

As used herein, “autoantibody” is an antibody made by an individual's immune system that is directed against the individual's own proteins acting as antigens.

By the terms “AD-related autoantibody” and “AD-related autoantibody biomarker” is meant an autoantibody that is associated with the presence of AD-related pathology. An AD-related autoantibody is made by the body in response to the damaged brain tissue caused by the presence of AD-related pathology. A “corresponding AD-related autoantibody” or “cognate AD-related autoantibody” means an AD-related autoantibody that specifically binds to a protein antigen target or epitope binding fragment thereof.

By the terms “PD-related autoantibody” and “PD-related autoantibody biomarker” is meant an autoantibody that is associated with the presence of PD-related pathology. A PD-related autoantibody is made by the body in response to the damaged brain tissue caused by the presence of PD-related pathology. A “corresponding PD-related autoantibody” or “cognate PD-related autoantibody” means a PD-related autoantibody that specifically binds to a protein target or epitope binding fragment thereof.

When referring to a “panel of autoantibodies” or a “panel of autoantibody biomarkers” what is meant is a plurality of autoantibodies that are binding to the protein targets described herein and in the methods, being detected.

As used herein, the term “protein target” refers to any full-length protein, non-full-length protein (e.g., full-length protein from which 1 or more amino acids have been removed), peptide, or epitope binding fragment that specifically binds to an AD-related autoantibody biomarker or PD-related autoantibody biomarker present in a biological fluid (e.g., blood, serum) from a subject or that binds indiscriminately to any antibody with a kappa light chain. Protein targets include antigens and epitope binding fragments thereof. AD-related protein targets used in the experiments described below are listed in Table 1. Protein targets MGC31944 and MGC31936 are isoforms (a protein isoform is a member of a set of highly similar proteins that originate from a single gene or gene family and may perform the same, similar, or unique biological functions). PD-related protein targets that can be used in the methods described herein are listed in Table 2.

By the term “protein target set” is meant a set of protein targets.

As used herein, the term “antigen” means a molecule that is specifically recognized and bound by an antibody. The protein targets described herein are antigens.

As used herein, “protein” and “polypeptide” are used synonymously to mean any peptide-linked chain of amino acids, regardless of length or post-translational modification, e.g., glycosylation or phosphorylation.

By the term “epitope binding fragments thereof” is meant any non-full-length protein or peptide that elicits a unique, specific immune response. When referring to a particular protein, the term includes autoantibody binding fragments thereof. Epitope binding fragments of a given protein target can be identified/determined using conventional immunological and binding assays, or through predictive sequence analysis software.

As used herein the term “substrate” means any solid phase to which the protein targets are directly or indirectly attached or coupled, such as a microtiter or microassay plate, slide, beads, microfluidics device, etc.

By the term “immunocomplex” is meant an AD- or PD-related autoantibody bound to a protein target or epitope binding fragment thereof.

As used herein “covariate” means an independent variable that can influence the outcome of a given statistical trial. Covariates include characteristics of a subject. In the methods and kits described herein, covariates can function as variables in a randomForest® decision, e.g., age as a continuous variable. In some embodiments, cognitive complaint can be a discrete variable.

By the term “AD score” is meant a value generated by an algorithm (e.g., a classification algorithm such as randomForest®) that indicates if the subject is likely, or not likely, to have AD-related pathology. The AD score can be a numerical percent, and in some embodiments can be a numerical probability ratio output, e.g., a ratio and percent that are outputs of a probability score. A PD score is generated in the same way as an AD score.

As used herein the term “classification algorithm” means any supervised learning technique that is used to identify the category of new observations on the basis of training data. Examples of a classification algorithm include machine learning algorithms, such as randomForest® Machine learning methods include statistical analysis, classification, etc.

By the term “predetermined threshold score” is meant a threshold score that is a cutoff value. For example, the predetermined threshold score can be static at 0.50 which is equivalent to 50%. In this example, if a subject's AD or PD score is 50% and above, the subject has a higher likelihood of AD- or PD-related pathology, and if the AD or PD score is below 50%, the subject has a lower likelihood of AD- or PD-related pathology.

By “normalization control” is meant a control that allows for the direct comparison of a patient's sample against knowns, compensating for variances in biology, run conditions, and manufactured batches.

By the term “conjugated” is meant when one molecule or agent is physically or chemically coupled or adhered or attached to another molecule or agent.

The terms “agent” and “therapeutic agent” as used herein refer to a chemical entity or biological product, or combination of chemical entities or biological products, administered to a subject to treat a disease or condition (e.g., AD, PD). Examples of agents include small molecule drugs and biologics.

The terms “patient,” “subject” and “individual” are used interchangeably herein, and mean a subject to be treated, diagnosed, and/or to obtain a biological sample from. Subjects include, but are not limited to, humans, non-human primates, horses, cows, sheep, pigs, rats, mice, dogs, and cats. A human in need of AD or PD diagnosis and/or treatment or suspected of needing AD or PD treatment is an example of a subject. A human who is at risk for AD or PD is another example of a subject.

As used herein, the terms “treatment” and “therapy” are defined as the application or administration of a therapeutic agent or therapeutic agents to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

All publications, patent applications, and patents mentioned herein are incorporated by reference in their entireties. In the case of conflict, the present specification, including definitions, will control. The particular embodiments discussed below are illustrative only and not intended to be limiting.

### EXAMPLES

The present invention is further illustrated by the following examples. The examples are provided for illustration only and should not be construed as limiting the scope of the invention in any way.

### Example 1—Early Detection of AD-Related Pathology Using a Multi-Disease Diagnostic Platform Employing Autoantibodies as Blood-Based Biomarkers

The experiments described here were performed to determine the utility of a panel of autoantibody biomarkers and a set of protein targets as described herein for detecting the presence of AD-related pathology along the early AD continuum, including at pre-symptomatic [an average of 4 years before the transition to mild cognitive impairment (MCI)/AD)], prodromal AD (MCI), and mild-moderate AD stages. A total of 328 serum samples from multiple cohorts, including ADNI subjects with confirmed pre-symptomatic, prodromal, and mild-moderate AD, were screened using Luminex xMAP® technology to predict the probability of the presence of AD-related pathology. A panel of protein targets used to detect their corresponding (cognate) autoantibody biomarkers present in serum, with age as a covariate, was evaluated using randomForest® and Receiver Operating Characteristic (ROC) curves. The panel of eight protein targets and corresponding autoantibody biomarkers predicted the probability of the presence of AD-related pathology with 81.0% accuracy and an area under the curve (AUC) of 0.84 (95% CI=0.78-0.91). Inclusion of age as a parameter to the model improved the AUC (0.96; 95% CI=0.93-0.99) and overall accuracy (93.0%). These results demonstrate that protein targets MGC31944 or MGC31936, HSH2D, GCDH, CCL19, LGALS1 (Galectin-1), DNAJC8, and ICAM-4 that are immobilized on Luminex magnetic beads (combined with anti-human anti-IgG kappa light chain antibody also immobilized on Luminex magnetic beads) and which bind to their corresponding (cognate) autoantibody biomarkers, can be used as an accurate, non-invasive, inexpensive, and widely accessible diagnostic screener for detecting AD-related pathology at pre-symptomatic and prodromal AD stages that could aid clinicians in diagnosing AD.

### Methods

### Study Population

Banked serum samples were obtained from independent cohorts collected from participants enrolled in clinical studies [ADNI, New Jersey Institute for Success Aging's (NJISA) Memory Assessment Program (MAP), and the Parkinson's Study Group] and from commercial sources. Serum from 64 confirmed pre-symptomatic AD participants, 71 with MCI due to AD with confirmed low CSF Aβ42 levels, and 24 with mild or moderate AD dementia were obtained from ADNI. Twenty-six additional MCI and 7 AD patient samples were obtained from the NJISA MAP Program (Stratford, NJ). Sera from 106 healthy, non-demented control subjects were obtained from Reprocell USA Inc. (Beltsville, MD). Twelve early-stage PD samples were obtained from the Parkinson's Study Group (Boston, MA). Eighteen stage 0-2 breast cancer serum samples were obtained from Asterand Bioscience, Inc. (Detroit, MI). All blood samples were handled using standard procedures. Demographic characteristics of the study population are listed in Table 3. The use of serum samples in this study was approved by the Rowan University Institutional Review Board (Pro2016001175 and Pro2012002275).

TABLE 3
Subject demographics.
Subject Demographics
Case Demographics (n = 192)

| | ADNI Pre- | ADNI | ADNI Mild- | Other | |
|---|---|---|---|---|---|
| | symptomatic | Prodromal | moderate | Cohort | All |
| | AD | AD (MCI) | AD | MCI/AD | Cases |
| | (n = 64) | (n = 71) | (n = 33) | (n = 24) | (n = 192) |
| Age Avg. | 76 (±6) | 73 (±8) | 74 (±7) | 75 (±9) | 75 (±7) |
| (Std. Dev.) | | | | | |
| Sex (Male %) | 59.4% | 54.9% | 30.3% | 58.3% | 52.6% |
| Ethnicity | | | | | |
| Asian (%) | 1.6% | 4.2% | 0.0% | NA | 2.5% |
| Black (%) | 7.8% | 1.4% | 0.0% | NA | 3.8% |
| Hispanic (%) | 1.6% | 2.8% | 0.0% | NA | 1.9% |
| White (%) | 89.1% | 91.5% | 100.0% | 100.0% | 91.8% |
| ApoE | | | | | |
| Proteotype | | | | | |
| E2/E3 (%) | 6.3% | 1.4% | 0.0% | NA | 3.1% |
| E2/E4 (%) | 3.1% | 0.0% | 0.0% | NA | 1.3% |
| E3/E3 (%) | 54.7% | 29.6% | 29.2% | NA | 39.6% |
| E3/E4 (%) | 29.7% | 53.5% | 41.7% | NA | 42.1% |
| E4/E4 (%) | 6.3% | 15.5% | 29.2% | NA | 13.8% |
| MMSE Avg. | 29 (±1) | 27 (±2) | 24 (±2) | NA | 27 (±2) |
| (Std. Dev.) | | | | | |
| CSF Aβ42 | 182 (±56) | 135 (±32) | 141 (±45) | NA | 152 (±48) |
| Avg. (Std. | | | | | |
| Dev.) | | | | | |
| CSF Tau | 78 (±35) | 119 (±53) | 108 (±42) | NA | 104 (±49) |
| Avg. (Std. | | | | | |
| Dev.) | | | | | |
| CSF pTau | 31 (±17) | 44 (±15) | 38 (±12) | NA | 39 (±17) |
| Avg. (Std. | | | | | |
| Dev.) | | | | | |

Control Demographics (n = 136)

| | Cognitively | Non- | | |
|---|---|---|---|---|
| | Normal | neurodegenerative | Neurodegenerative | |
| | Control | Control - Breast | Control - PD | All Controls |
| | (n = 106) | Cancer (n = 18) | (n = 12) | (n = 136) |
| Age Avg. | 56 (±12) | 47 (±6) | 60 (±9) | 55 (±11) |
| (Std. Dev.) | | | | |
| Sex (Male %) | 50.9% | 0.0% | 33.0% | 42.6% |
| Ethnicity | 100.0% | 100.0% | 100.0% | 100.0% |
| (White) | | | | |

The number of individuals (n), age, gender, and race are listed for each case and control group.
For ADNI samples, ApoE proteotype, MMSE, and CSF Aβ42, Tau, and pTau are included as additional data.

### Pre-Analytical Serum Processing

Blood collection and serum pre-processing was similar among all cohorts. ADNI, Durin Technologies Inc., Reprocell, and Parkinson's Study Group blood samples were collected in red top tubes (BD 367820), allowed to sit at room temperature for at least 15 minutes to clot, centrifuged, aliquoted, and frozen at −80° C. Asterand Bioscience Inc. samples were collected in red tiger top serum separator tubes (BD 367985), allowed to sit at room temperature for at least 30 minutes to clot, centrifuged, aliquoted, and frozen at −20° C. or cooler.

### Antigens (Protein Targets)

The following recombinant human protein target antigens were coupled to Luminex xMAP® Microspheres: a custom made IGL-MGC31944 (Custom R & D/Biotechne), HSH2D (Custom R & D/Biotechne), GCDH (MyBioSource—Catalog #MBS8249095), CCL19 (MyBioSource—Catalog #MBS203647), LGALS1 (Galectin-1) (Novus—Catalog #NBP2-76255), DNAJC8 (Novus—Catalog #H00022826-P01), ICAM-4 (Abnova-Catalog #H00003386-G01), and a recombinant Rabbit Anti-Human Kappa Light Chain antibody (Abcam-Catalog #ab 195576) (Table 4). Proteins with buffers incompatible with the coupling chemistry were washed in 1×PBS and concentrated using protein concentrators (Pierce-Catalog #88516) before coupling.

TABLE 4
Panel of AD-related protein targets.

| Database ID | Protein Name |
|---|---|
| BC022098.1 | cDNA clone MGC: 31944 IMAGE: 4878869 |
| | (IGL-MGC31944) |
| NM_032855.1 | hematopoietic SH2 domain containing (HSH2D) |
| NM_006274.3 | chemokine (C-C motif) ligand 19 (CCL19) |
| NM_201998.1 | Glutaryl-Coenzyme A dehydrogenase, nuclear |
| | gene encoding mitochondrial protein, |
| | transcript variant 1 (GCDH) |
| NM_002305.4 | Lectin, galactoside-binding, soluble, 1 |
| | (galectin 1) (LGALS1) |
| NM_014280.3 | DnaJ homolog subfamily C member 8 (DNAJC8) |
| NM_001544.5 | Intercellular adhesion molecule 4 |
| | (Landsteiner-Wiener blood group) |
| | transcript variant 1 (ICAM4) |
| n/a | Anti-Human Kappa Light Chain Antibody |

Database identifiers and descriptions of the seven AD-related protein targets and the anti-human Kappa Light Chain antibody protein target.

### Microsphere-Protein Target Coupling

Microsphere-protein target coupling was carried out using the Luminex xMAP® Antibody Coupling (AbC) Kit (40-50016) according to manufacturer's recommendations. All antigenic proteins (protein targets) were coupled at 25 pmol/million beads. Coupled beads corresponded to Luminex xMAP® bead regions 12 (IGL-MGC31944/BC022098.1), 18 (HSH2D), 29 (Anti-Kappa), 33 (GCDH), 36 (CCL19), 44 (LGALS1), 46 (DNAJC8), and 48 (ICAM4). Protein target coupling was confirmed by testing serial dilutions of an in-house control human serum standard and/or antigen-specific antibodies.

### Assay Procedure

2,500 beads/region were combined with 50 μl bead mix in each well of a Costar 96 Well Plate (Catalog #3912). 50 μl of participant serum, diluted 1/50 in phosphate-buffered saline (PBS TBN), was added to each well and mixed for 30 minutes at 37° C. with shaking on an Eppendorf Thermomixer FP at 650 rpm. Samples were washed 3× with 80 μl PBS-TBN using a BioTek 405 TS plate washer. 100 μl of Phycoerythrin (PE) antibody (0.5 mg/ml) was added to each well and incubated for 20 minutes at 37° C. with shaking. Samples were again washed 3× with 80 μl of PBS-TBN, resuspended in 100 μl PBS-TBN, and analyzed using a Luminex FlexMap3D instrument with count volume set to 50 μl and the minimum bead count set at 50. All samples were run in duplicate and averaged to obtain final working values. Samples with a Coefficient of Variation (CV %) greater than 15% were discarded. Final inter- and intra-assay CV % were calculated at 10.4% and 4.9%, respectively.

### Statistical and Graphical Analysis

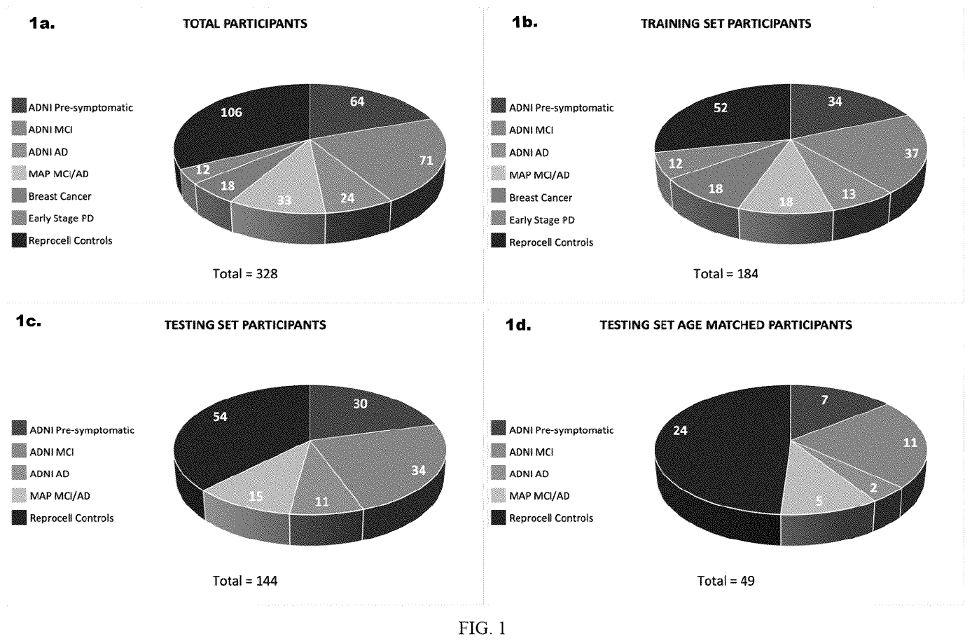

AD and healthy non-cognitively impaired control subjects were randomly split into Training and Testing Sets such that both sets contained participants of roughly equal age and sex distribution. All PD and breast cancer subjects were relegated to the Training Set. The Training Set consisted of 34 pre-symptomatic AD, 37 MCI, and 13 mild and moderate AD from ADNI, 12 MCI and 6 AD from the NJISA MAP cohort, 52 non-demented controls, as well as 12 PD and 18 breast cancer samples to represent neurodegenerative and non-neurodegenerative disease controls, respectively. The remaining samples were relegated to the Testing Set and included 30 pre-symptomatic AD, 34 MCI, 11 mild and moderate AD from ADNI, 14 MCI and 1 AD from the NJISA MAP cohort, and 54 non-demented controls. Sample grouping between the Training and Testing Sets can be found in FIG. 1(a)-1(d).

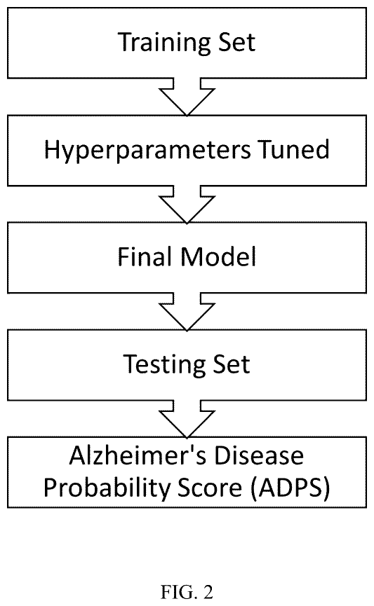

The predictive probability model using eight protein targets (cDNA clone MGC: 31944 IMAGE: 4878869, HSH2D, GCDH, CCL19, LGALS1, ICAM4, DNAJC8, anti-IgG Kappa light chain antibody) and age as a covariate for all stages of AD represented was developed and optimized using only subjects from the Training Set and randomForest®; no testing datasets were used to tune hyperparameters or optimize the final randomForest® predictive model in any way (RF; v 4.6-10) in R (v 4.0.0) (The R Foundation for Statistical Computing) (Breiman L (2001) RandomForests. Machine Learning 45, 5-32). The final model derived from the Training Set subjects was used to predict the probability of AD-related pathology in the Testing Set subjects. This probability was reported as the Alzheimer's disease probability score (ADPS). An overview of the process can be found in FIG. 2. Receiver Operating Characteristic (ROC) curves were calculated using R packages ROCR (v 1.0-11) and pROC (v 1.1.18) [44], and the probability of being disease-positive is reported as a function of ROC sensitivity and specificity for each model. Additional R packages used in data analysis and visualization included ggplot 2 (v.3.3.6), and epiR (v 2.0.52).

### Calculation of the Alzheimer's Disease Probability Score (ADPS)

Samples in each of the Testing Sets were classified as either AD or a control using a percent probability output ranging from 0-100, known as the Alzheimer's disease probability score (ADPS). The ADPS represents the fraction of trees in the forest that vote for a certain class (i.e. AD or control). Using the ADPS, classification as either AD or control was based on a specific cutoff threshold derived using ROC curves to determine the optimal cutoff value corresponding to the largest Youden's J Statistic (sensitivity+specificity−1). All samples with a probability score above the threshold were classified as AD, and all samples falling below the threshold were classified as controls.

### RESULTS

Serum IgG autoantibody (aAB) biomarkers can be used to detect AD-related pathology in patients with pre-symptomatic, prodromal, and more advanced AD

Previous studies using human protein microarrays described a small group of aAB biomarkers that could be used in an assay to identify patients with prodromal AD (MCI), confirmed with low CSF Aβ42 levels, with high overall accuracy (DeMarshall et al., Alzheimers Dement (Amst) 3, 51-62). The latter is consistent with the presence of brain amyloidosis and a high likelihood of later progression to AD. Here, this assay was migrated to the Luminex magnetic bead platform, and a panel of seven previously identified blood-borne IgG aAB biomarkers comprising four prodromal AD (MCI) biomarkers (cDNA clone MGC: 31944 IMAGE: 4878869, HSH2D, GCDH, CCL19), and three mild-moderate AD biomarkers (LGALS1, ICAM4, DNAJC8) from earlier studies (Table 4), were detected via binding to their respective cognate (corresponding) protein targets immobilized on the magnetic beads. The goal was to determine if patients at multiple points along the early AD continuum could be distinguished from non-demented controls in a single test. This study had 328 participants, including 64 cognitively normal participants who later progressed to MCI/AD (here referred to as pre-symptomatic AD), 71 with prodromal AD (MCI), and 24 with mild-moderate AD, all from ADNI, along with 33 MCI/AD sera obtained from another memory clinic (NJISA MAP cohort) and 106 non-demented controls. Relative levels of the aAB biomarkers in sera were measured using their respective protein targets immobilized on Luminex magnetic beads and a customized Luminex xMAP® magnetic bead assay. Samples were separated into Training and Testing Sets, each containing roughly equal numbers of samples from patients spanning multiple stages of AD as well as non-demented controls, and were evaluated for the presence of AD-related pathology using randomForest®. Additionally, the Training Set contained 12 early-stage PD samples as neurodegenerative controls, and 18 breast cancer samples as non-neurodegenerative controls in the total control group to aid in the development of the diagnostic model for detection of early AD-related pathological processes.

Using randomForest® to evaluate Training Set samples (n=184; 102 cases, 82 controls), a diagnostic model was created utilizing the seven autoantibody biomarkers alone, with an out-of-bag (OOB) error of 22.3%. This model was then applied to Testing Set subjects to determine the overall classification accuracy. Subjects in the Testing Set (n=144; 90 cases, 54 controls), which included pre-symptomatic, prodromal, and mild-moderate AD subjects as cases as well as healthy, non-demented controls, were classified as either positive for AD-related neuropathology or negative (controls), with an overall classification accuracy of 81.0%, sensitivity of 80.0%, specificity of 81.0%, positive predictive value (PPV) of 88.0%, and a negative predictive value (NPV) of 71.0%, indicating that autoantibody biomarker levels are concordant with the presence of ongoing AD-related pathology, as was confirmed in the ADNI cohort. The diagnostic utility of this panel of seven AD-related autoantibody biomarkers alone was also evaluated using ROC curve analysis of Testing Set subjects (FIG. 1(a)-1(d)). The ROC area under the curve (AUC) for this comparison was 0.84 (95% CI=0.78-0.91). Diagnostic sensitivity, specificity, PPV, and NPV for the AD-related autoantibody biomarkers when used alone to evaluate Testing Set subjects can be found in Table 5.

TABLE 5
Diagnostic sensitivity, specificity, PPV, and NPV for the AD-related autoantibody
biomarkers when used alone to evaluate Testing Set subjects
Testing Set
Subjects

| | | | AUC | Sensitivity | Specificity | PPV % | NPV % | |
|---|---|---|---|---|---|---|---|---|
| | | | (95% | (95% | (95% | (95% | (95% | Accuracy |
| | n | Threshold | CI) | CI) | CI) | CI) | CI) | % |
| Markers + | 49 | 0.65 | 0.97 | 1 | 0.92 | 0.93 | 1 | 96.0 |
| Age | | | (0.93, 1) | (0.87, 1) | (0.74, | (0.77, | (0.85, | |
| (age- | | | | | 0.98) | 0.98) | 1.00) | |
| matched | | | | | | | | |
| controls) | | | | | | | | |
| Markers + | 144 | 0.56 | 0.96 | 0.92 | 0.94 | 0.97 | 0.88 | 93.0 |
| Age | | | (0.93, 0.99) | (0.85, | (0.85, | (0.90, | (0.77, | |
| (non- | | | | 0.96) | 0.98) | 0.99) | 0.94) | |
| age- | | | | | | | | |
| matched | | | | | | | | |
| controls) | | | | | | | | |
| Markers | 144 | 0.48 | 0.84 | 0.80 | 0.81 | 0.88 | 0.71 | 81.0 |
| | | | (0.78, 0.91) | (0.71, | (0.90.69, | (0.79, | (0.59, | |
| | | | | 0.87) | 0) | 0.93) | 0.81) | |

Diagnostic utility (Testing Set subjects only) of the autoantibody biomarkers alone, and with age as a covariate for predicting the probability of the presence of AD-related pathology in cases compared to controls.
Area under the curve (AUC) values at 95% confidence were generated using ROC curve analysis.
Threshold values were derived using ROC curves to find the optimal cutoff value corresponding to the largest Youden's J Statistic.
Overall accuracy, sensitivity, specificity, PPV, and NPV are derived from probability data with 95% confidence intervals generated using the Wilson score method for binominal proportions

### Inclusion of Age as a Covariate Improves Model Performance and Detection of Ongoing AD-Related Pathology

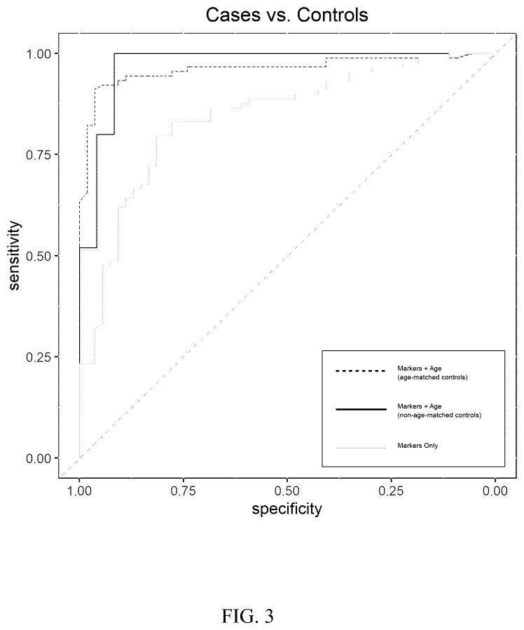

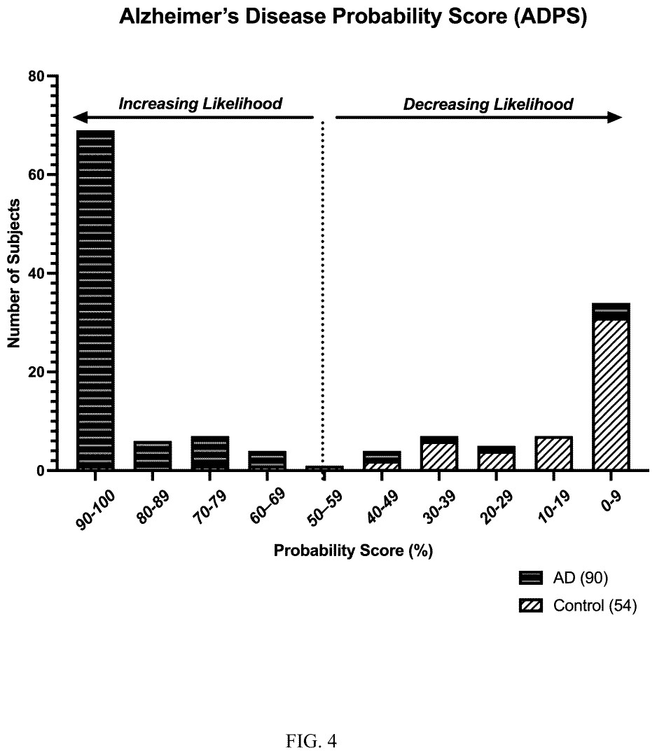

Age has been a long-established risk factor for AD. Here, whether adding subject age as a covariate in randomForest® analysis significantly improved model performance and overall diagnostic accuracy was examined. Addition of age as a continuous variable was found to improve the diagnostic model, resulting in a decrease of the OOB error from 22.3% to 8.2%. Overall accuracy in the Testing Set subjects was improved from 81.0% to 93.0%, and the ROC AUC from 0.84 to 0.96 (95% CI=0.93-0.99), and had a sensitivity of 92.0%, specificity of 94.0%, PPV of 97.0%, and NPV of 88.0% (Table 4). ROC AUC comparisons with the addition of age as a covariate are shown in FIG. 3. Furthermore, using randomForest® analysis, an Alzheimer's disease probability score (ADPS) ranging from 0-100 was calculated for predicting the likelihood of the presence of ongoing AD-related pathology as indicated by the panel of seven autoantibody biomarkers that are detected by the panel of protein targets as described herein and accompanying age covariate data. Based on this model, a score of 56 or greater indicates a higher likelihood for the presence of AD-related pathology, while a score of 55 or lower indicates a reduced likelihood. The probability score distribution for Testing Set subjects is shown in FIG. 4

### Performance of the aAB Biomarker Panel in an Age-Matched Cohort

Due to the progressively increasing prevalence of AD in aging adults, as well as the fact that neurodegenerative changes associated with this disease can begin up to two decades before the onset of clinical symptoms, the task of identifying healthy and appropriately age-matched control subjects lacking early stages of AD pathology can be fraught with potential error. This is particularly problematic for tests that are highly sensitive. In the Testing Set described above, a control population that was roughly twenty years younger than the AD sample population was purposely used to minimize the likelihood of the presence of early AD-related pathological changes in the controls. To demonstrate that the chosen panel of aAB biomarkers is not simply classifying patient samples largely based on age, a closer age-matched control population was tested by creating an additional Testing Set utilizing control samples from the original Testing Set that were obtained from individuals aged 60 years and older. Subjects in this new age-matched Testing Set (n=49; 25 cases, 24 controls) included pre-symptomatic, prodromal, and mild-moderate AD samples with an average age of 71 as well as healthy, non-demented controls with an average age of 66. These samples were classified as either positive for AD-related pathology or controls using the panel of aAB biomarkers and age as a covariate, with an overall classification accuracy of 96.0%, sensitivity of 100.0%, specificity of 92.0%, PPV of 93.0%, and NPV of 100.0% (Table 5). This demonstrates the high sensitivity and specificity of the aAB biomarker panel when used with closely age-matched subjects, with results comparable to the overall accuracy obtained using the non-age-matched Testing Set described above. The diagnostic utility of detecting and measuring these aAB biomarkers using the protein targets described herein was also evaluated using ROC curve analysis (FIG. 1(a)-1(d)). The ROC area under the curve (AUC) for this comparison was 0.97 (95% CI=0.93-1).

### aAB Biomarkers can Detect the Presence of AD-Related Pathology in Prodromal and Later Stages of AD

To further confirm the utility of the panel of autoantibody biomarkers in accurately detecting early stages of ongoing AD-related pathology as well as later stages, how many prodromal AD subjects with low CSF Aβ42 levels and mild-moderate AD samples in the Testing Set were correctly classified compared to controls was evaluated. Using randomForest® logic derived from Training Set samples based on the chosen aAB biomarkers and age covariate, 31 of 34 prodromal and all 11 mild-moderate ADNI AD samples were correctly classified. Additionally, 10 of 13 prodromal and 2 of 2 mild-moderate AD subjects from an additional cohort, the Memory Assessment Program at the New Jersey Institute for Successful Aging, were also correctly classified using the same strategy. This data suggests that the overall diagnostic strategy of including the seven aAB biomarkers plus age as a covariate is robust, correctly identifying 87.2% of all confirmed prodromal AD and 100% of mild-moderate AD subjects across two independent cohorts with high overall accuracy, sensitivity, and specificity. Importantly, sera from all prodromal AD participants obtained from ADNI came from individuals with low CSF Aβ42 levels, consistent with the presence of ongoing early-stage brain amyloidosis, a hallmark pathological feature of early stages of AD.

aAB Biomarkers Detect the Presence of Early AD-Related Pathological Processes in Subjects with Confirmed Pre-Symptomatic AD

ADNI criteria of pre-symptomatic AD include those who initially enrolled as cognitively normal participants, but who several years later had transitioned to confirmed MCI due to AD or more advanced stages of AD dementia. ADNI criteria for normal controls include a) the absence of subjective cognitive concerns that are not due to the normal aging process, b) within normative expectation performance on cognitive screeners (MMSE and CDR) and tests (Logical Memory) (see the Alzheimer's Disease Neuroimaging Initiative website for cut-off scores), and c) no report of functional decline. It was then asked if this diagnostic strategy, using the same panel of seven aAB biomarkers along with age as a covariate, was sensitive enough to detect the presence of ongoing AD-related pathology at an even earlier pathological stage, i.e., before the onset of observable clinical symptoms. To address this, sera from 64 ADNI participants at or near baseline who were originally diagnosed as cognitively normal based on neuropsychological assessments and normal CSF Aβ42 levels, but who later transitioned to either prodromal AD or a more advanced mild-moderate AD were obtained. These participants were classified as pre-symptomatic AD, and individuals in this group transitioned from cognitively normal to a diagnosis of MCI due to AD within an average of 48.3 months (median=47.5 months) after entry into the study as cognitively normal controls. Again, using the randomForest® logic derived from Training Set samples based on the seven chosen aAB biomarkers and the age covariate, 29 of 30 pre-symptomatic ADNI participants in the Testing Set were correctly identified as having AD pathology, demonstrating a 96.6% sensitivity for pre-symptomatic detection of AD-related pathological processes (Table 6).

TABLE 6
Testing Set Subjects

| Correctly | | ADNI | ADNI | ADNI | NJISA | NJISA | |
|---|---|---|---|---|---|---|---|
| Classified | n | Pre-symptomatic | MCI | MMAD | MAP MCI | MAP AD | NDC |
| Markers + Age | 49 | 7/7 | 11/11 | 2/2 | 4/4 | 1/1 | 22/24 |
| (age-matched | | | | | | | |
| controls) | | | | | | | |
| Markers + Age | 144 | 29/30 | 31/34 | 11/11 | 10/13 | 2/2 | 51/54 |
| (non-age- | | | | | | | |
| matched | | | | | | | |
| controls) | | | | | | | |
| Markers | 144 | 23/30 | 29/34 | 11/11 | 8/13 | 2/2 | 45/54 |

Breakdown of the probability score analysis in the Testing Set subjects using the panel of seven autoantibody biomarkers and age covariate in each AD-related pathological group and the non-demented control group.

Testing of these seven aAB biomarkers using the protein targets described herein resulted in four main findings. First, this set of aAB biomarkers identified individuals with prodromal AD and mild-moderate AD as positive for AD-related pathology and distinguished them from cognitively normal controls with high overall accuracy. Second, inclusion of age as a covariate significantly improved overall diagnostic performance at all disease stages tested. Third, the panel of seven autoantibody biomarkers that were detected and measured using the protein targets described herein also achieved detection of AD-related pathology with high overall accuracy in pre-symptomatic AD participants who originally enrolled in ADNI as cognitively normal controls, but a few years later transitioned to prodromal or more advanced AD with confirmed AD pathology. The fact that the same seven aAB biomarkers worked well for identifying pre-symptomatic, prodromal, and mild-moderate disease stages when patients at different stages of the disease were combined into a single large group supports a scenario where it is not necessary to establish independent cutoff values for each cohort or stage of the disease. This enables production of a single test that can detect the presence of AD-related pathology within a relatively broad range of the early AD continuum.

The results described herein have also shown that the detection of serum aAB biomarkers is not platform-specific; migration of the aAB biomarker technology from human protein microarrays of previous studies to a Luminex magnetic bead-based platform, while retaining comparable performance, was successful.

In conclusion, the Luminex magnetic bead-based analytical platform described here accurately identified the presence of early AD-related pathology in individuals with pre-symptomatic, prodromal and mild-moderate AD based on detection of seven disease-associated IgG aAB biomarkers in a single blood sample using a panel of seven cognate protein targets and anti-kappa light chain antibody immobilized on the beads as described herein. Addition of age as a covariate to the model employing the seven aAB biomarkers contributed to the excellent performance of this blood test. The development of a relatively noninvasive, accurate blood test for use in early detection of AD-related pathology at pre-symptomatic, prodromal, and mild-moderate stages is a significant advancement in the field given that detection and measurement of the seven aAB biomarkers (1) can reliably distinguish individuals with normal vs. abnormal cognitive function and predict clinical decline even in those who are asymptomatic at baseline; (2) are minimally invasive, inexpensive, and usable in frontline or community primary care settings for screening a general population; and (3) could serve as a surrogate measure for predicting outcomes in AD and AD-related dementia treatment trials. It may enable more informed determinations of which patients in the primary care settings should undergo further, more extensive neuropsychological evaluations and more invasive and costly neuroimaging (MRI and PET) and CSF diagnostic procedures. This would be of great benefit to patients and clinical practice since early treatment has the greatest potential to bend the curves on clinical outcomes. The ability to detect AD-related pathology at earlier pre-symptomatic and prodromal (MCI) stages will allow participants to be enrolled earlier in targeted clinical trials, and greatly facilitate monitoring of AD progression, including in those under treatment.

### Example 2—Detection of Pre-Symptomatic Ad

The results described herein show that the diagnostic strategy of using the same protein targets along with age as a covariate was sensitive enough to detect autoantibody biomarkers associated with the presence of AD pathology at the pre-symptomatic AD stage, i.e., before the onset of observable clinical symptoms. Sera from 79 ADNI participants who were originally diagnosed as cognitively normal upon baseline neuropsychological assessment and normal CSF Aβ42 levels, but later transitioned within an average of 49.5 months to either prodromal or a more advanced mild-moderate AD, were tested. Again, using the same eight protein targets (the, randomForest® logic derived from Training Set samples and an age covariate, 37 of 40 (92.5%) pre-symptomatic subjects in the Testing Set were correctly identified as having AD pathology. This is the first known test to accurately identify the presence of AD-related pathology in pre-symptomatic AD participants several years before the onset of clinical symptoms, and earlier than currently available neuropsychological assessments can identify subjective cognitive decline. It is well-known that low CSF Aβ42 levels occur primarily at or near MCI, and thus represents a relatively narrow window of pathological progression along the AD continuum. The ability to detect AD pre-symptomatically in subjects lacking telltale changes in CSF Aβ42 levels suggests that the serum aAB biomarker levels increase before changes in CSF Aβ42 levels occur, and that these fluid biomarkers reflect different aspects/stages of amyloid pathology. Unlike with CSF Aβ42 levels, the results described herein show that the same seven aAB biomarkers in serum can be used to detect the presence of AD-related pathology at pre-symptomatic, prodromal (MCI), and mild-moderate AD stages. Clinical experience shows that patients who arrive for the first time at their doctors with a cognitive and memory complaint can be at widely varying stages of AD, ranging from subtle cognitive impairment—to MCI—to mild-moderate AD. The fact that the same protein targets worked well for identifying the autoantibody biomarkers associated with pre-symptomatic, prodromal and mild-moderate disease stages when patients at different stages of the disease were combined into a single large testing group provides a single test that can detect the presence of AD pathology within a broad range of the early-stage AD continuum.

### Example 3—Use of the Multi-Disease Diagnostic Platform to Guide Patient Evaluation and Treatment

The protein targets used to detect autoantibody biomarkers associated with the presence of AD-related pathology described herein are used in frontline or community primary care settings for screening a general population for the presence of AD-related pathology; and serve as a surrogate measure for monitoring disease progression and predicting outcomes in AD and AD-related dementia treatment trials. The autoantibody biomarkers detected using the protein targets, including when combined with covariate data, enable more informed determinations of which patients in primary care settings should undergo further, more extensive neuropsychological evaluation and more invasive and costly neuroimaging (MRI and PET) and CSF diagnostic procedures. This is of great benefit to patients and clinical practice since early treatment has the greatest potential to bend the curves on clinical outcomes. The ability to detect the presence of AD-related pathology at earlier pre-symptomatic and prodromal (MCI) stages allows participants to be enrolled earlier in targeted clinical trials and greatly facilitates monitoring of AD progression, including those under treatment.

### Example 4—Use of the Multi-Disease Diagnostic Platform for Detection of Additional Diseases

For neurodegenerative diseases in which the cell types involved in the pathology closely resemble one another, autoantibody biomarkers need to be disease-specific. Based on the results described herein showing that the eight protein targets are capable of distinguishing AD subjects from those with other neurodegenerative (e.g., PD) and non-neurodegenerative (e.g., breast cancer) diseases with exceptionally high accuracy, when patients present with MCI not associated with AD, such as drug side-effects, poor vascular perfusion of the brain, and chronic depression, the panel of 8 protein targets described herein, are used to accurately distinguish these patients from those with MCI due to ongoing AD, which could prompt physicians to search for other causes of their MCI.

### Example 5—Pre-Symptomatic Detection and Monitoring of Ad Using a Multi-Disease Diagnostic Platform Employing Autoantibodies as Blood-Based Biomarkers and Luminex XMAP® Technology

Evidence for the universal presence of serum autoantibodies and their potential diagnostic utility for detection of AD and other neurodegenerative diseases has been demonstrated previously. It is well known that AD-related pathological changes in the brain can begin up to a decade before patients experience telltale symptoms, yet pre-symptomatic detection remains an unmet goal. In the present study, the utility of a panel of protein targets that detect a panel of AD-related autoantibodies found in patients' serum is capable of detecting AD at the earliest points along the AD continuum, including pre-symptomatic AD, years before the onset of symptoms, and prodromal AD (mild cognitive impairment, MCI).

Using a customized panel of eight protein targets that detect autoantibody biomarkers and Luminex xMAP® technology, sera from ADNI subjects with confirmed pre-symptomatic AD or MCI were screened to demonstrate both pre-symptomatic and prodromal AD detection. A panel of eight protein targets (seven of which specifically bind to seven autoantibody biomarkers with increased titer in MCI and pre-symptomatic AD relative to controls) was evaluated using both randomForest® and Receiver Characteristic Operating curves for their ability to distinguish diseased subjects from age- and sex-matched controls, as well as from individuals with other neurodegenerative and non-neurodegenerative diseases.

Results showed that the panel of eight protein targets affixed to Luminex magnetic beads was capable of differentiating patients with MCI from corresponding age- and sex-matched controls with high overall accuracy, sensitivity, and specificity. These protein targets were also capable of identifying cognitively normal subjects who later converted to MCI and AD, years before the clinical onset of their symptoms. This protein target set readily distinguished MCI and pre-symptomatic AD subjects from Parkinson's disease and breast cancer subjects, demonstrating excellent disease specificity.

Results demonstrate the utility of this custom Luminex xMAP® blood-based protein target panel as an accurate, non-invasive, and inexpensive diagnostic screener, not only for the detection of AD-related pathology at prodromal AD, but also at earlier stages of pathology, several years before the first onset of telltale clinical symptoms. This approach can be used as a multi-disease diagnostic and disease-staging strategy, useful for a multitude of neurodegenerative diseases including, as examples, early-stage AD and PD, as well as Multiple Sclerosis and amyotrophic lateral sclerosis (ALS).

### Example 6—Disease-Associated Autoantibodies in the Serum and their Potential Utility as Diagnostic Biomarkers of Pre-Symptomatic Disease

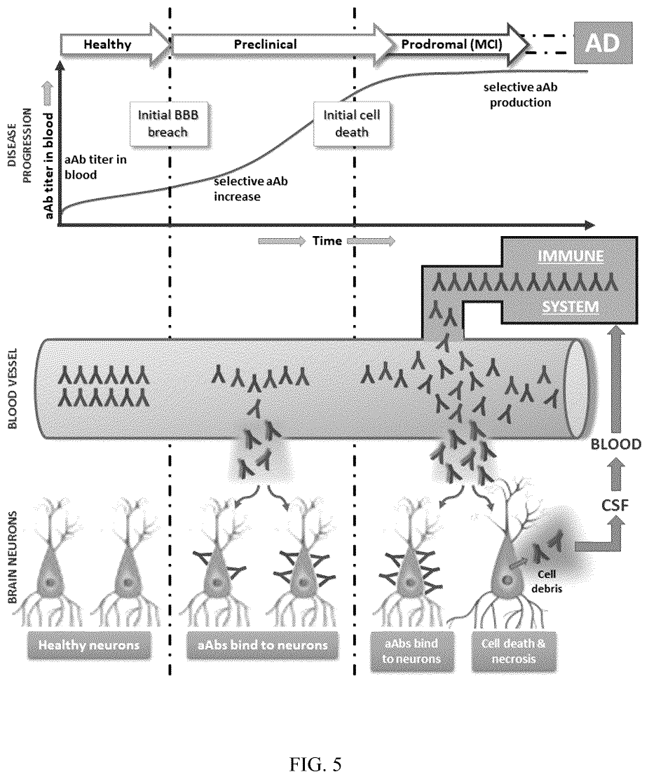

Referring to the diagram of FIG. 5, the pre-symptomatic stage in AD progression can last up to a decade or more before symptoms appear. Over time, these pathological changes escalate to the prodromal stage of disease progression where neurons become overburdened by chronic receptor stripping via endocytosis and local amyloid deposition, resulting in increasing levels of cell death and necrosis. During this entire process, cellular debris from dead neurons is released into brain parenchyma, enters the cerebrospinal fluid, and makes it way into the blood, activating the immune system and eliciting the production of corresponding autoantibodies. Autoantibodies exhibiting the most dramatic and consistent changes compared to controls are selected as the most useful biomarkers of AD. The methods, systems and kits described herein are the first blood-based diagnostic test to accurately detect pre-symptomatic AD, up to 5 years before the emergence of telltale clinical symptoms.

### Example 7—the Anti-Kappa Bead Enables Generation of Igg Standard Curves Used to Estimate Total Igg Levels in the Blood

In the methods, kits and systems described herein, the fraction of IgG in a biological sample that contains the kappa light chain is detected and measured. To detect and measure this, an anti-kappa light chain antibody is attached to a bead. This can be used to generate IgG standard curves. In an example of such a method: anti-kappa antibody (purified and commercially available) is attached to Luminex beads; anti-kappa antibody on the bead reacts with any IgGs in serum that possess a kappa chain; anti-kappa antibody does not interfere with protein target MGC31944 that is an IgG antibody that has a Lambda chain instead of kappa; purified IgG standards (commercially available) are used to generate standard curves as a reference in embodiments of the methods described herein; the resulting Standard Curve Equation is used to calculate levels of total IgG in each patient's serum sample; and the curve can also be used to determine levels of the other autoantibody markers (by applying the curve equation to the other IgG signals).

### Example 8—Early-Stage Detection and Monitoring of Parkinson'S Disease Using a Multi-Disease Diagnostic Platform Employing Autoantibodies as Blood-Based Biomarkers

The protein targets listed in Table 2, in combination with anti-human anti-IgG kappa light chain antibody, were used in the detection and quantification of the levels of PD-related autoantibodies in the biological sample that are associated with the presence of PD-related pathology. Using protein targets MARK1, PUSL1, IL20, and CCL19 that specifically bind to PD-related autoantibody biomarkers in combination with the anti-human anti-IgG kappa light chain antibody, the presence of PD-related pathology can be detected at an early disease stage. In the experiments described below, sera from a total of 398 subjects, including 103 early-stage PD subjects derived from the Deprenyl and Tocopherol Antioxidative Therapy of Parkinsonism (DATATOP) study, were screened with human protein microarrays (Invitrogen), to predict the probability of the presence of PD-related pathology. A panel of four protein targets were evaluated using randomForest®. Use of the protein targets MARK1, PUSL1, IL20, CCL 19 and anti-human anti-IgG kappa light chain antibody predicted a patient's probability of a PD diagnosis with an overall accuracy of 87.9%, sensitivity of 94.1%, and specificity of 85.5%. These protein targets also differentiated patients with early-stage PD from those with more advanced (mild-moderate) PD with an accuracy of 97.5%, and could distinguish subjects with early-stage PD from those with other neurological diseases including Alzheimer's disease with an accuracy of 97.0%, and multiple sclerosis with an accuracy of 96.3%, as well as a non-neurological disease, breast cancer, with an accuracy of 97.5%.

Methods of detecting early PD-related pathology are generally carried out as the methods for detecting early AD-related pathology described herein. For example, a PD score is generated in the same manner as an AD score as described herein.

### Other Embodiments

Any improvement may be made in part or all of the kits, systems, and method steps. All references, including publications, patent applications, and patents, cited herein are hereby incorporated by reference in their entireties. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

1. A method for detecting Alzheimer's Disease (AD)-related pathology in a subject, the method comprising:
(a) obtaining an immunoglobulin-containing biological sample from the subject;
(b) providing a protein target set comprising protein targets MGC31944 or MGC31936, HSH2D, GCDH, CCL19, LGALS1 (Galectin-1), DNAJC8, ICAM-4, and anti-human anti-IgG kappa light chain antibody, and/or epitope binding fragments thereof, wherein the protein targets and/or epitope binding fragments thereof are attached to a substrate,
(c) contacting the biological sample with the protein target set under conditions under which each of MGC31944 or MGC31936, HSH2D, GCDH, CCL19, LGALS1 (Galectin-1), DNAJC8, and ICAM-4, or epitope binding fragments thereof, forms an immunocomplex with a corresponding AD-related autoantibody biomarker if present in the biological sample, and the anti-human anti-IgG kappa light chain antibody forms immunocomplexes with all IgG antibodies having a kappa light chain in the biological sample;
(d) adding a detectable label to the immunocomplexes such that the immunocomplexes are labeled;
(e) measuring the levels of the labeled immunocomplexes to generate levels of AD-related autoantibody biomarkers and a total IgG antibody level in the biological sample;
(f) obtaining at least one covariate data set generated from the subject; and
(g) determining an AD score for the subject by inputting the levels of the subject's AD-related autoantibody biomarkers, total IgG antibody level, and the covariate data into a classification algorithm configured to (1) determine therefrom an AD score, and (2) output the AD score.

2. The method of claim 1, further comprising at least one step selected from the group consisting of: analyzing at least one covariate of the subject to generate covariate data, receiving the AD score, and detecting the presence of AD-related pathology in a subject with early stage AD if the subject's AD score is equal to or greater than a predetermined threshold score at or above which there is a likelihood that a subject has ongoing AD-related pathology.

3. A method for detecting the presence of Parkinson's Disease (PD)-related pathology in a subject with early-stage PD, the method comprising:
(a) obtaining an immunoglobulin-containing biological sample from the subject;
(b) providing a protein target set comprising protein targets MARK1, PUSL1, IL20, CCL19, and anti-human anti-IgG kappa light chain antibody, and/or epitope binding fragments thereof, wherein the protein targets and/or epitope binding fragments thereof are attached to a substrate,
(c) contacting the biological sample with the protein target set under conditions under which each of MARK1, PUSL1, IL20, CCL19, or epitope binding fragments thereof, forms an immunocomplex with a corresponding PD-related autoantibody biomarker if present in the biological sample, and the anti-human anti-IgG kappa light chain antibody forms immunocomplexes with all IgG antibodies having a kappa light chain in the biological sample;
(d) adding a detectable label to the immunocomplexes such that the immunocomplexes are labeled;
(e) measuring the levels of the labeled immunocomplexes to generate levels of PD-related autoantibody biomarkers and a total IgG antibody level in the biological sample;
(f) analyzing at least one covariate of the subject to generate covariate data; and
(g) determining an PD score for the subject by: (A) inputting the levels of the subject's PD autoantibody biomarker, total IgG antibody level, and the covariate data into a classification algorithm configured to (1) determine therefrom an PD score, and (2) output the PD score.

4. The method of claim 3, further comprising at least one step selected from the group consisting of: analyzing at least one covariate of the subject to generate covariate data, receiving the PD score, and detecting that the subject has early-stage PD-related pathology if the subject's PD score is equal to or greater than a predetermined threshold score at or above which there is a likelihood that a subject has ongoing PD-related pathology.

5. The method of claim 1, wherein the biological sample is blood or serum.

6. The method of claim 1, wherein the subject has AD-related pathology consistent with pre-symptomatic AD, prodromal AD (mild cognitive impairment), mild-moderate AD, or does not have AD-related pathology.

7. The method of claim 3, wherein the subject has PD-related pathology consistent with early-stage PD, or does not have PD-related pathology.

8. The method of claim 1, wherein the biological sample is blood or serum.

9.-14. (canceled)

15. The method of claim 1, wherein measuring the total IgG antibody level in the biological sample is a normalization control, and the detectable label comprises labeled anti-human secondary antibody.

16. The method of claim 1, wherein measuring the total IgG antibody level in the biological sample is a normalization control, and the detectable label comprises labeled anti-human secondary antibody.

17. The method of claim 16, wherein the anti-human secondary antibody is labeled with a fluorescent label, and wherein in step d), measuring the levels of the labeled immunocomplexes comprises measuring fluorescence.

18. The method of claim 15, wherein the anti-human secondary antibody is labeled with a fluorescent label, and wherein in step d), measuring the levels of the labeled immunocomplexes comprises measuring fluorescence.

19. The method of claim 1, wherein the at least one covariate is age, and wherein the classification algorithm is a machine learning algorithm.

20. The method of claim 1, wherein the at least one covariate is age, and wherein the classification algorithm is a machine learning algorithm.

21. The method of claim 2, further comprising at least one of: conducting at least one additional corroborating diagnostic test of the subject, and treating the subject if the method determines that the subject has AD-related pathology.

22. (canceled)

23. The method of claim 4, further comprising at least one of: conducting at least one additional corroborating diagnostic test of the subject, and treating the subject if the method determines that the subject has early-stage PD.

24. (canceled)

25. A kit for detecting the presence of AD-related pathology in a subject, the kit comprising:
(a) a protein target set comprising protein targets MGC31944 or MGC31936, HSH2D, GCDH, CCL19, LGALS1 (Galectin-1), DNAJC8, ICAM-4, and anti-human anti-IgG kappa light chain antibody, and/or epitope binding fragments thereof, wherein the protein targets and/or epitope binding fragments thereof are attached to a substrate comprising a plurality of beads, each bead attached to only one type of protein target, and/or epitope binding fragment thereof, of the protein targets,
(b) a multi-well plate wherein each well of the multi-well plate contains a bead mix and a portion of the plurality of beads that are each attached to only one type of protein target, and/or epitope binding fragment thereof, of the protein targets, wherein each protein target, and/or epitope binding fragment thereof, is represented in each well of the multi-well plate;
(c) at least one control; and
(d) assay reagents for detection of: i) immunocomplexes formed by binding of each protein target or epitope binding fragment thereof to a corresponding AD-related autoantibody if present in the subject's immunoglobulin-containing biological sample, and ii) immunocomplexes formed by binding of the anti-human anti-IgG kappa light chain antibody to all IgG antibodies having a kappa light chain in the subject's biological sample,
wherein the assay reagents comprise a detectable label for labeling the formed immunocomplexes.

26. A kit for detecting the presence of PD-related pathology in a subject with early-stage PD, the kit comprising:
(a) a protein target set comprising protein targets MARK1, PUSL1, IL20, CCL19, and anti-human anti-IgG kappa light chain antibody, and/or epitope binding fragments thereof, wherein the protein targets and/or epitope binding fragments thereof are attached to a substrate comprising a plurality of beads, each bead attached to only one type of protein target, and/or epitope binding fragment thereof, of the protein targets,
(b) a multi-well plate wherein each well of the multi-well plate contains a bead mix and a portion of the plurality of beads that are each attached to only one type of protein target, and/or epitope binding fragment thereof, of the protein targets, wherein each protein target, and/or epitope binding fragment thereof, is represented in each well of the multi-well plate;
(c) at least one control; and
(d) assay reagents for detection of: i) immunocomplexes formed by binding of each protein target or epitope binding fragment thereof to a corresponding PD-related autoantibody biomarker if present in the subject's immunoglobulin-containing biological sample, and ii) immunocomplexes formed by binding of the anti-human anti-IgG kappa light chain antibody to all IgG antibodies having a kappa light chain in the subject's biological sample,
wherein the assay reagents comprise a detectable label for labeling the formed immunocomplexes.

27.-30. (canceled)

31. A kit for detecting the presence of AD-related pathology in a subject, the kit comprising:
(a) a protein target set comprising protein targets MGC31944 or MGC31936, HSH2D, GCDH, CCL19, LGALS1 (Galectin-1), DNAJC8, ICAM-4, and anti-human anti-IgG kappa light chain antibody, and/or epitope binding fragments thereof, wherein the protein targets and/or epitope binding fragments thereof are attached to a substrate comprising a microfluidics device,
(b) at least one control; and
(c) assay reagents for detection of: i) immunocomplexes formed by binding of each protein target or epitope binding fragment thereof to a corresponding AD autoantibody biomarker if present in the subject's immunoglobulin-containing biological sample, and ii) immunocomplexes formed by binding of the anti-human anti-IgG kappa light chain antibody to all IgG antibodies having a kappa light chain in the subject's biological sample,
wherein the assay reagents comprise a detectable label for labeling the formed immunocomplexes.

32. A kit for detecting the presence of PD-related pathology in a subject with early-stage PD, the kit comprising:
(a) a protein target set comprising protein targets MARK1, PUSL1, IL20, CCL19, and anti-human anti-IgG kappa light chain antibody, and/or epitope binding fragments thereof, wherein the protein targets and/or epitope binding fragments thereof are attached to a substrate comprising a microfluidics device,
(b) at least one control; and
(c) assay reagents for detection of: i) immunocomplexes formed by binding of each protein target or epitope binding fragment thereof to a corresponding PD-related autoantibody if present in the subject's immunoglobulin-containing biological sample, and ii) immunocomplexes formed by binding of the anti-human anti-IgG kappa light chain antibody to all IgG antibodies having a kappa light chain in the subject's biological sample,
wherein the assay reagents comprise a detectable label for labeling the formed immunocomplexes.

33.-41. (canceled)


SEQUENCE LISTING (12 sequences)

SEQ ID NO: 1 - protein, 237 aa, Homo sapiens
MAWSPLLLTLLAHCTGSWAQSVLTQPPSVSGAPGQRVTISCTGSSSNIGAGYDVHWYQQLPGTAPKLLIYGNSNRPSGVPDRFSGSKSGTSASLAITGLQAEDEADYYCQSYDYSLSASGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 2 - protein, 237 aa, Homo sapiens
MAWSPLLLTLLAHCTGSWAQSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGFVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 352 aa, Homo sapiens
MTEAGKLPLPLPPRLDWFVHTQMGQLAQDGVPEWFHGAISREDAENLLESQPLGSFLIRVSHSHVGYTLSYKAQSSCCHFMVKLLDDGTFMIPGEKVAHTSLDALVTFHQQKPIEPRRELLTQPCRQKDPANVDYEDLFLYSNAVAEEAACPVSAPEEASPKPVLCHQSKERKPSAEMNRITTKEATSSCPPKSPLGETRQKLWRSLKMLPERGQRVRQQLKSHLATVNLSSLLDVRRSTVISGPGTGKGSQDHSGDPTSGDRGYTDPCVATSLKSPSQPQAPKDRKVPTRKAERSVSCIEVTPGDRSWHQMVVRALSSQESKPEHQGLAEPENDQLPEEYQQPPPFAPGYC

SEQ ID NO: 4 - protein, 98 aa, Homo sapiens
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 5 - protein, 438 aa, Homo sapiens
MALRGVSVRLLSRGPGLHVLRTWVSSAAQTEKGGRTQSQLAKSSRPEFDWQDPLVLEEQLTTDEILIRDTFRTYCQERLMPRILLANRNEVFHREIISEMGELGVLGPTIKGYGCAGVSSVAYGLLARELERVDSGYRSAMSVQSSLVMHPIYAYGSEEQRQKYLPQLAKGELLGCFGLTEPNSGSDPSSMETRAHYNSSNKSYTLNGTKTWITNSPMADLFVVWARCEDGCIRGFLLEKGMRGLSAPRIQGKFSLRASATGMIIMDGVEVPEENVLPGASSLGGPFGCLNNARYGIAWGVLGASEFCLHTARQYALDRMQFGVPLARNQLIQKKLADMLTEITLGLHACLQLGRLKDQDKAAPEMVSLLKRNNCGKALDIARQARDMLGGNGISDEYHVIRHAMNLEAVNTYEGTHDIHALILGRAITGIQAFTASK

SEQ ID NO: 6 - protein, 135 aa, Homo sapiens
MACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

SEQ ID NO: 7 - protein, 253 aa, Homo sapiens
MAASGESGTSGGGGSTEEAFMTFYSEVKQIEKRDSVLTSKNQIERLTRPGSSYFNLNPFEVLQIDPEVTDEEIKKRFRQLSILVHPDKNQDDADRAQKAFEAVDKAYKLLLDQEQKKRALDVIQAGKEYVEHTVKERKKQLKKEGKPTIVEEDDPELFKQAVYKQTMKLFAELEIKRKEREAKEMHERKRQREEEIEAQEKAKREREWQKNFEESRDGRVDSWRNFQANTKGKKEKKNRTFLRPPKVKMEQRE

SEQ ID NO: 8 - protein, 271 aa, Homo sapiens
MGSLFPLSLLFFLAAAYPGVGSALGRRTKRAQSPKGSPLAPSGTSVPFWVRMSPEFVAVQPGKSVQLNCSNSCPQPQNSSLRTPLRQGKTLRGPGWVSYQLLDVRAWSSLAHCLVTCAGKTRWATSRITAYKPPHSVILEPPVLKGRKYTLRCHVTQVFPVGYLVVTLRHGSRVIYSESLERFTGLDLANVTLTYEFAAGPRDFWQPVICHARLNLDGLVVRNSSAPITLMLAWSPAPTALASGSIAALVGILLTVGAAYLCKCLAMKSQA

SEQ ID NO: 9 - protein, 795 aa, Homo sapiens
MSARTPLPTVNERDTENHTSVDGYTEPHIQPTKSSSRQNIPRCRNSITSATDEQPHIGNYRLQKTIGKGNFAKVKLARHVLTGREVAVKIIDKTQLNPTSLQKLFREVRIMKILNHPNIVKLFEVIETEKTLYLVMEYASGGEVFDYLVAHGRMKEKEARAKFRQIVSAVQYCHQKYIVHRDLKAENLLLDGDMNIKIADFGFSNEFTVGNKLDTFCGSPPYAAPELFQGKKYDGPEVDVWSLGVILYTLVSGSLPFDGQNLKELRERVLRGKYRIPFYMSTDCENLLKKLLVLNPIKRGSLEQIMKDRWMNVGHEEEELKPYTEPDPDFNDTKRIDIMVTMGFARDEINDALINQKYDEVMATYILLGRKPPEFEGGESLSSGNLCQRSRPSSDLNNSTLQSPAHLKVQRSISANQKQRRFSDHAGPSIPPAVSYTKRPQANSVESEQKEEWDKDVARKLGSTTVGSKSEMTASPLVGPERKKSSTIPSNNVYSGGSMARRNTYVCERTTDRYVALQNGKDSSLTEMSVSSISSAGSSVASAVPSARPRHQKSMSTSGHPIKVTLPTIKDGSEAYRPGTTQRVPAASPSAHSISTATPDRTRFPRGSSSRSTFHGEQLRERRSVAYNGPPASPSHETGAFAHARRGTSTGIISKITSKFVRRDPSEGEASGRTDTSRSTSGEPKERDKEEGKDSKPRSLRFTWSMKTTSSMDPNDMMREIRKVLDANNCDYEQKERFLLFCVHGDARQDSLVQWEMEVCKLPRLSLNGVRFKRISGTSIAFKNIASKIANELKL

SEQ ID NO: 10 - protein, 303 aa, Homo sapiens
MSSAPASGSVRARYLVYFQYVGTDFNGVAAVRGTQRAVGVQNYLEEAAERLNSVEPVRFTISSRTDAGVHALSNAAHLDVQRRSGRPPFPPEVLAEALNTHLRHPAIRVLRAFRVPSDFHARHAATSRTYLYRLATGCHRRDELPVFERNLCWTLPADCLDMVAMQEAAQHLLGTHDFSAFQSAGSPVPSPVRTLRRVSVSPGQASPLVTPEESRKLRFWNLEFESQSFLYRQVRRMTAVLVAVGLGALAPAQVKTILESQDPLGKHQTRVAPAHGLFLKSVLYGNLGAASCTLQGPQFGSHG

SEQ ID NO: 11 - protein, 176 aa, Homo sapiens
MKASSLAFSLLSAAFYLLWTPSTGLKTLNLGSCVIATNLQEIRNGFSEIRGSVQAKDGNIDIRILRRTESLQDTKPANRCCLLRHLLRLYLDRVFKNYQTPDHYTLRKISSLANSFLTIKKDLRLCHAHMTCHCGEEAMKKYSQILSHFEKLEPQAAVVKALGELDILLQWMEETE

SEQ ID NO: 12 - protein, 98 aa, Homo sapiens; identical to SEQ ID NO: 4